(19) United States
(12) Patent Application Publication
(10) Pub. No.: US 2009/0117635 A1
(43) Pub. Date: May 7, 2009

(54) Process for Integrating Cellulose and Starch Feedstocks in Ethanol Production

(75) Inventors: Clifford Bradley, Missoula, MT (US); Robert Kearns, Melrose, MT (US)

Correspondence Address: MORGAN, LEWIS & BOCKIUS, LLP, ONE MARKET SPEAR STREET TOWER, SAN FRANCISCO, CA, 94105, US

(73) Assignee: ENERGY ENZYMES, INC., Cupertino, CA (US)

(21) Appl. No.: 12/264,884

(22) Filed: Nov. 4, 2008

Related U.S. Application Data
(60) Provisional application No. 60/985,408, filed on Nov. 5, 2007.
(60) Provisional application No. 60/985,430, filed on Nov. 5, 2007.
(60) Provisional application No. 60/985,452, filed on Nov. 5, 2007.
(60) Provisional application No. 61/021,211, filed on Jan. 15, 2008.
(60) Provisional application No. 61/024,339, filed on Jan. 29, 2008.
(60) Provisional application No. 61/097,169, filed on Sep. 15, 2008.

(51) Int. Cl. C12P 7/10

(52) U.S. Cl. 435/165

(57) ABSTRACT

The present invention is directed to process integrating cellulose and starch feedstocks to produce ethanol.

### CROSS REFERENCE TO RELATED APPLICATIONS

This application claims the benefit of, and priority to, U.S. Provisional Patent Application Ser. Nos. 60/985,452, 60/985,430, and 60/985,408 filed on Nov. 5, 2007, 61/021,211, filed on Jan. 15, 2008, 61/024,339 filed on Jan. 29, 2008, and 61/097,169 filed on Sep. 15, 2008, the entire disclosures of both of which are hereby incorporated by reference in their entireties.

### FIELD OF THE INVENTION

The present invention is directed to process of integrating cellulose and starch feed stocks to produce ethanol.

### BACKGROUND OF THE INVENTION

One of the renewable alternative energy sources are biofuels converted from biomass. Of many of substitutes to gasoline, one of the most generally recognized substitutes which could be made available in significant quantities in the near future is alcohol, and in particular, ethanol. For example, there are currently many outlets in the United States and throughout the world which sell a blend of gasoline and about 10%- to 20% ethanol (commonly called “gasohol”) which can be used as a fuel in conventional automobile engines. Furthermore, ethanol can be blended with additives to produce a liquid ethanol-based fuel, with ethanol as the major component, which is suitable for operation in most types of engines. Ethanol can be produced from almost any material which either exists in the form of, or can be converted into, a fermentable sugar. There are many natural sugars available for fermentation, but carbohydrates such as starch and cellulose can be converted into fermentable sugars which then are fermented into ethanol.

Starch is one of the world's most abundant renewable raw materials. One answer to the need for alternative reproducible fuels is to convert this very abundant material at low cost into fermentable sugars as feedstock for fermentation to ethanol. A recent review article describes a long history of published research in production and characterization of raw starch hydrolysis enzymes. Robertson et al, J. Agric. And Food Chem 54:353-365 (2006), herein incorporated by reference.

Currently, most ethanol is produced from starch in corn grain using amylase enzymes to degrade the starch to fermentable sugars. In general, while the corn grain is used in the production of ethanol, the remainder of the corn biomass, i.e., the leaves and stalks, is seldom unused because of the cost in degrading the leaves and stalks comprising lignins and cellulose, generally in the form of lignocellulose, to fermentable sugars. The lignocellulose in the stalks and leaves of corn biomass represents a tremendous source of untapped energy that goes unused because of the difficulty and cost of converting it to fermentable sugars. There are also abundant sources of cellulose that could be tapped into to produce ethanol, such as paper mill waste and grass.

The present invention provides a process for integrating cellulosic and grain feedstocks in a manner that takes advantage of the low cost of cellulosic materials while improving the efficiency of distillation.

### SUMMARY OF THE INVENTION

The invention provides a method of producing ethanol, comprising: providing a fermented beer from a cellulose, hemicellulose fermentation; mixing said fermented beer with a starch source to form a second mash; mixing said second mash with enzymes (e.g. amylase enzymes) and yeast and/or ethanol producing bacteria; and incubating for a period of fermentation time under a second temperature between 20 to 78° C. to produce ethanol. In some embodiments, the fermented cellulose beer contains less than 7% (w/w) ethanol. In some embodiments, the fermented cellulose beer contains about 2.5 to 6% (w/w) ethanol. In some embodiments, the starch in the second mash is un-gelatinized. In some embodiments, the second temperature is about 35° C. In some embodiments, the method is carried at a temperature lower than the boiling point of ethanol. In some embodiments, the incubating step comprises simultaneously hydrolysis and fermentation. In some embodiments, the ethanol produced is less than 15%. In some embodiments, the method further comprises collecting the ethanol. In some embodiments, the method further comprises distilling said ethanol.

In some embodiments, the cellulose fermentation comprising: providing a first mash that is adjusted to pH 4 to 6; mixing said first mash with enzymes (e.g. amylase enzymes) and yeast and/or ethanol producing bacteria; and incubating for a period of fermentation time under a first temperature between 20 to 40° C. to produce said fermented beer. In some embodiment, the incubating is conducted at pH 4.5 to 5.0° C. In some embodiments, the cellulose and or hemicellulose is first mixed with enzymes to produce fermentable sugars, then incubated with yeast in a separate step to produce ethanol. The enzyme step is adjusted to optimum for the enzyme preparation used, generally pH 4 to 6 and temperature up to 50° C.

### BRIEF DESCRIPTION OF THE DRAWINGS

FIG. 1 depicts a typical cellulosic ethanol production process with alkaline pretreatment.

FIG. 2 depicts an acid delignification cellulosic ethanol process.

FIG. 3 depicts a comparison of different starch hydrolysis process.

FIG. 4 depicts a simplified integrated process for ethanol production.

FIG. 5 depicts a system for ethanol production from cellulose and/or starch.

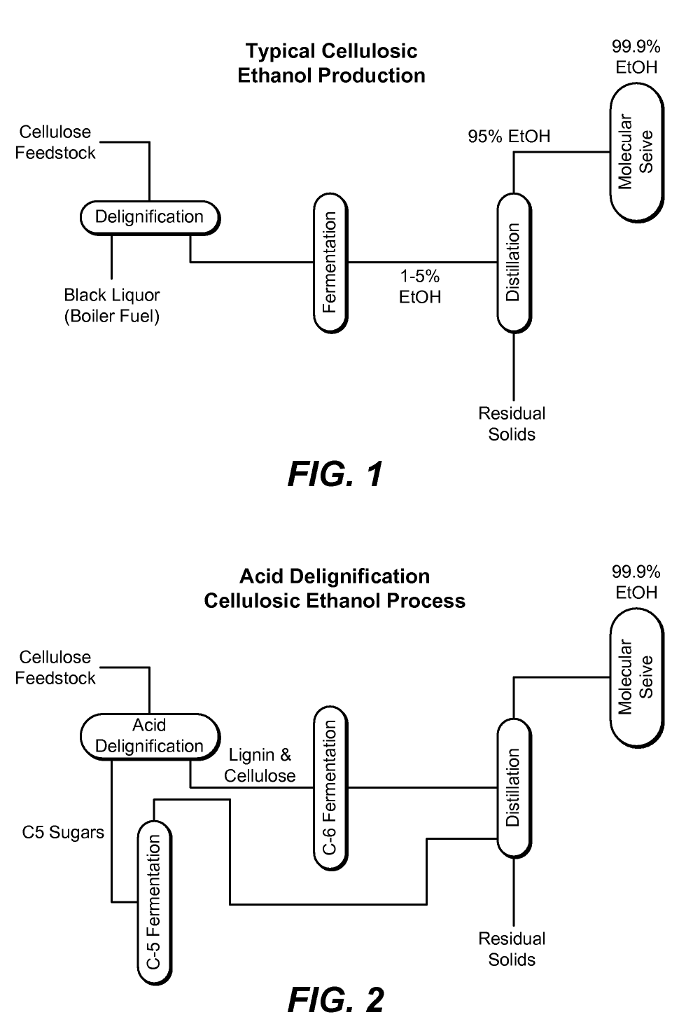

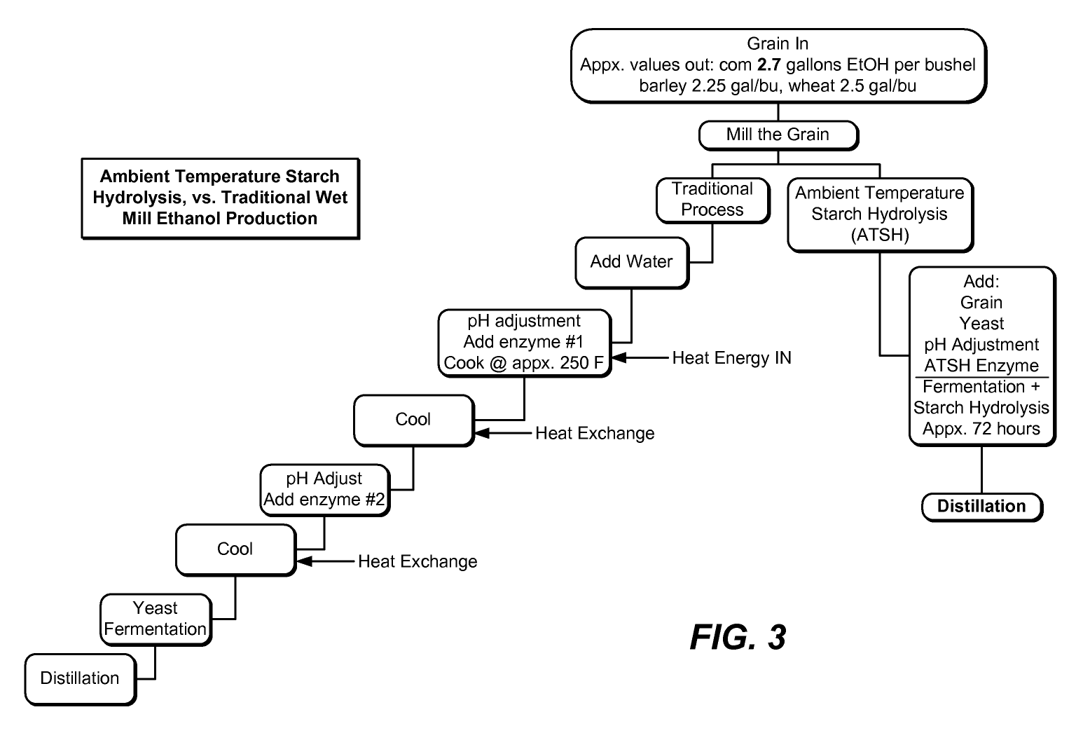

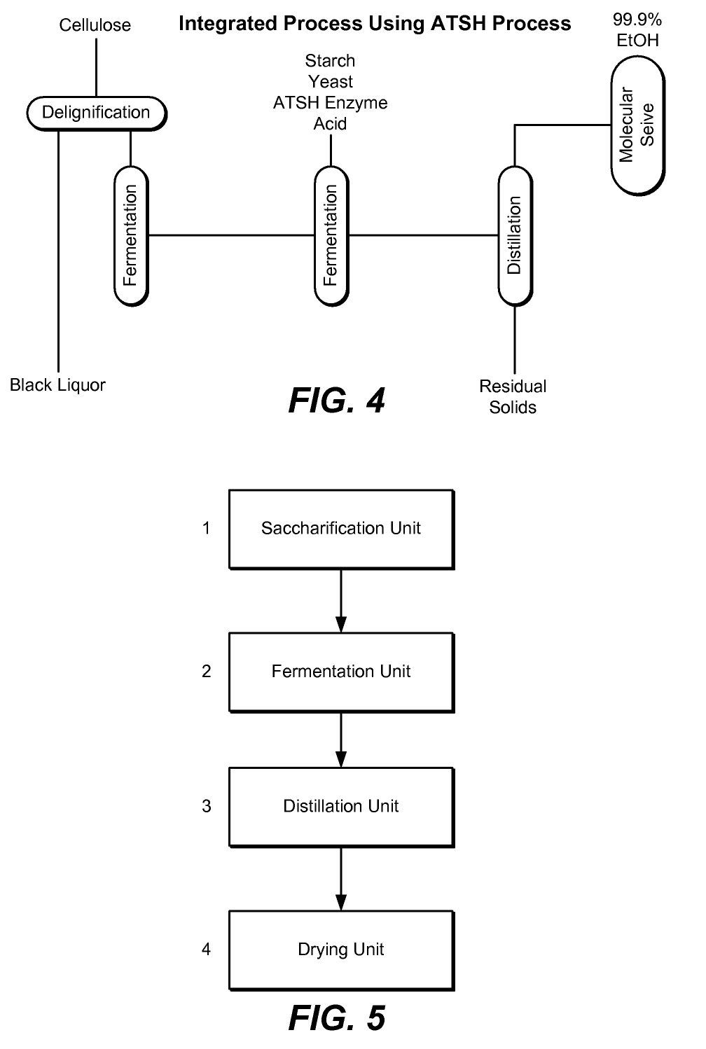

### DETAILED DESCRIPTION OF THE INVENTION

In general, the present invention relates to methods and compositions to produce ethanol that integrates the use of starch and cellulosic feedstocks in the same process.

In one aspect, the present invention provides a method of producing ethanol wherein cellulose and/or hemicellulose is hydrolyzed to produce fermentable sugar, which is fermented to produce a beer. This fermented beer then is mixed with a starch source to form a mash, which is then hydrolyzed and fermented to produce ethanol.

The ability to integrate cellulosic and starch feedstocks in ethanol production has important technical and economic advantages over the use of single feedstocks. Cellulosic feedstocks such as straw, corn stover, wood waste etc. may be low cost compared to grain or sugar feedstocks for ethanol production. However several factors may limit the economic advantages of these materials in ethanol production. First the low bulk density of these materials and limited availability in an area can result in inefficient and expensive transportation or limit the size of an ethanol plant. Second, the water sorption and viscosity of cellulosic materials limits the solids loading in enzymatic hydrolysis and fermentation. In practice, solids loading of greater than about 10% are generally too thick to effectively agitate or pump. As the final ethanol concentration in the fermentation is a function of the feedstock concentration, the low solids loading results in a low final ethanol concentration in the fermentation. Low ethanol concentrations increase the energy requirement and cost of distillation compared with starch based feedstocks. For example, with cellulosic feedstocks at 10% w/w initial solids, the final ethanol concentration will range from 3% to at most 5% w/w. Ethanol concentrations can range from to 10% to 12% w/w ethanol with grain feedstocks such as corn or barley, partially as a result of the higher solids loading in the fermentation slurry. Lower ethanol concentrations in the cellulosic slurries require more distillation energy per unit of ethanol recovered than starch based processes with higher ethanol concentrations.

An additional factor of economic importance is the fermentation of sugars derived from the hemicellulose fractions of lignocellulosic feedstocks. Hydrolysis of hemicellulose yields a mixture of five carbon sugars including xylose and arabinose as well as six carbon sugars such galactose which are not fermented to ethanol by the yeast Saccharomyces cerevisiae commonly used in commercial ethanol production. The species of yeast or bacteria that ferment hemicellulose derived sugars to ethanol typically have very low ethanol tolerance compared to conventional yeast. Yeast or bacteria that ferment sugars from hemicellulose hydrolysis typically have a maximum ethanol tolerance of 6% or less.

Integrating cellulose and starch feedstocks provides the opportunity to collect more feedstock over a shorter transportation radius allowing greater production in a region and greater economies of scale. For example, integrating both grain and grain straw as ethanol feedstocks allows larger scale ethanol production over a shorter feedstock transport radius in a region than either feedstock alone.

Integrating the cellulose and starch process essentially allows the same water to be used through the entire process, reducing the overall water requirement per gallon of ethanol produced. The integrated facility uses waste biomass from the cellulose process to generate energy for the process, improving the energy balance. Distillation costs are reduced.

The two key components to integrate the cellulose process with a starch process are: 1) using the beer from the cellulose fermentation as the mash makeup water for the starch process, and 2) using amylase enzyme preparations in the starch mash that hydrolyze ungelatinized raw starch granules.

The present invention uses the fermented beer from a cellulose fermentation containing ethanol as the fermentation water for a starch hydrolysis and fermentation. In some embodiments, the fermented beer from cellulose fermentation can contain up to 7% w/w ethanol. In this way the ethanol derived from the cellulose is blended with the grain starch derived ethanol to achieve a higher final concentration, in some embodiments up to 15% w/w. The combination of cellulose derived ethanol with starch derived ethanol leads to a higher final ethanol concentration, which translates into much more efficient distillation.

### I. Hydrolysis of Cellulose Material

In one aspect of the present invention, the ethanol production process starts with the hydrolysis of cellulose material. Any cellulose hydrolysis methods known in the art can be used in the present invention, and particularly, include the methods disclosed in U.S. application Ser. No. 61/021,211, filed Jan. 15, 2008, and the patent application titled “Process of producing ethanol using cellulose with enzymes generated through solid state culture” filed concurrently with this application by the same applicant, both are incorporated by reference in their entireties.

In some embodiments, cellulose is hydrolyzed chemically. In some embodiments, the cellulose is hydrolyzed with acid. In some embodiments, no enzyme is used in the cellulose hydrolysis.

In some embodiments, cellulose is hydrolyzed using enzymes, such as cellulase from commercial sources, such as Genencor or Novozymes, is used to hydrolyze the cellulose, as described in detail in the Examples.

In some embodiments, cellulase, hemicellulase enzymes produced by solid substrate culture of selected fungal strains is used.

The process of producing enzymes by growing a fungus on solid culture begins with selection of the proper fungus and substrate.

In one aspect, the present invention provides a strain of Trichoderma reesei (also known as Hypocrea jecorina) that can be used in methods of the invention. In another aspect, the present invention provides growth substrates and growing conditions that allow production of enzyme preparations using fungus, such as the strain of Trichoderma provided herein. The invention can be generally described as follows. The substrate is selected to provide nutrition for fungal growth and the physical structure of the solid substrate culture. The dry substrate is moistened with added water or a nutrient containing solution then steamed to adjust moisture and reduce contamination from indigenous microorganisms. The steamed substrate is inoculated with the desired fungus and loaded into a solid support growth chamber. The final moisture content of the substrate is such that the moisture is absorbed into the substrate and the substrate remains solid. The fungus grows on the substrate, utilizing it as a nutrient source, and at the same time producing the desired enzymes. This incubation time varies depending on the enzymes being produced. After the incubation, the whole culture is harvested to obtain the enzyme preparation. In some embodiments, the whole culture is used for converting cellulose to sugar and no additional purification of the enzymes is required. Alternatively the enzymes can be extracted and purified from the culture substrate. These enzyme preparations called, referred herein as SSC Cellulase, can be used in any process for enzymatic hydrolysis and or enzymatic hydrolysis and fermentation of lignocellulose. Generally, the preferred process is a simultaneous hydrolysis of cellulose and fermentation conducted at the upper temperature limit of the yeast, generally about 35° C. and the pH optima of the enzyme about pH 4.8. In another aspect, the present invention provides methods for fermenting sugar into ethanol.

The process of converting delignified lignocellulose to ethanol requires hydrolysis of both the cellulose and hemicellulose fractions and can be summarized as the following:

The first step is the hydrolysis of cellulose:

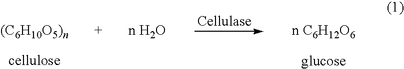

The second step is the conversion of glucose to ethanol:

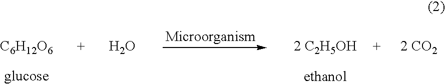

The first step of cellulose hydrolysis can be further broken into two steps:

One step is the conversion of cellulose to cellobiose:

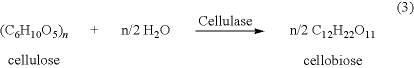

This is followed by a step of converting cellobiose to glucose:

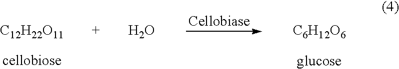

The process of hemicellulase (such as xylanse) hydrolysis followed by fermentation is the follows:

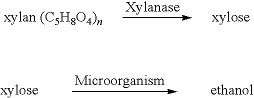

Thus, the present invention provides a process comprising enzyme production using solid substrate culture, the composition of an enzyme preparation containing multiple cellulase and hemicellulase activities and a second step to produce ethanol.

### A. Strain Selection

In one aspect of the present invention, a strain of Trichoderma reesei obtained from a public collection (USDA) is used for growth on cellulose at low pH. When grown in the SSC process as provided herein this strain produces a mixture of enzymes that hydrolyze the cellulose at an optimum pH of 4.8.

Trichoderma reesei is a mesophilic and filamentous fungus, the anamorph of Hypocrea jecorina. It has the capacity to secrete large amounts of cellulolytic enzymes (cellulases and hemicellulases). A detailed description of the Trichoderma reesei strain used in the present invention is provided in Example 1. The strain is ATCC-56765.

By “strain” herein is meant a genetic variant or subtype of a fungus. Thus, there is genotype and/or phenotype difference between a strain and the parent strain from which it is derived. The creation of a new strain can due to either naturally occurred mutations or artificially introduced mutations.

Other fungus that can produce cellulases and or hemicellulases may also be used in the present invention. Suitable fungal species include other strains of T. reesei, Aspergillus niger, A. phoenicis, A. oryzae, A awamori, Rhizopus oryzae, R. microsporus, Acidothermus cellulyticus and Trichoderma koningii, Trichoderma viride. T. harzianum, Fusarium oxysporum Penicillum pupurogenum Myceliophthora sp., Lentinous. Other suitable organisms are listed in Pandey et al., Current Science (Bangalore), 77(1):149-163 (1999), herein incorporated by reference.

### B. Solid Substrate Culture Technology

The instant application further provides solid substrate culture technology (sometimes referred to as solid state fermentation or solid state culture) to produce enzyme preparations capable of converting cellulose to glucose and hemicellulose to monomercic C5 and C6 sugars, including xylose, arabinose and galactose.

Conventional ethanol processes uses enzymes produced in liquid fermentation. In general, these are specific enzymes that are purified and concentrated. Furthermore, when more than one enzyme is used in an ethanol process (e.g., hydrolysis of cellulose to glucose), the individual enzymes are generally produced in separate liquid fermentation vessels. One of the primary costs of enzymes produced in liquid culture is the cost of concentrating the enzymes or separating the enzymes from the broth in which they are grown. The more liquid in the process, the higher transportation, storage and distillation costs. The present invention provides new methods of producing multiple enzymes in the same culture at high concentrations, and generally, does not require post-production purification.

Accordingly, the instant invention provides a solid substrate culture technology that results in enzyme preparations produced from one organism with high concentrations of multiple enzyme activities that work effectively in downstream ethanol production from cellulose.

By “solid substrate culture (SSC)” or “solid state fermentation (SSF)” herein is meant a culture wherein the organism is grown on the surface of a moist solid material where a majority (or in some cases, all) of the water is absorbed into the substrate material. Thus, there is a minimum amount of water or substantially no free water in the culture, which facilitates handling, and minimizes bacterial contamination among other things. The substrate material provides both the nutrients and physical support for the culture. The organism obtains oxygen from the air or from modified atmosphere introduced into the growth chamber. Depending on the composition and water sorbancy of the substrate, the substrate moisture can range from 30 to 90% w/w final moisture content. In the present invention the substrate moisture generally ranges from about 50 to 80% with an optimum about 65 to 70%.

Solid substrate culture is different from conventional liquid fermentation. In a liquid fermentation system, a microorganism is placed in a liquid environment that contains soluble nutrients. Air or oxygen is bubbled through the liquid using agitation or injection to dissolve oxygen in the liquid. Generally there is not any solid support media and the oxygen available to the organism is limited by the solubility of oxygen in water.

Solid culture technology has been around for over a hundred years. Most applications of solid culture technology involve the use of specific substrates or nutrients to achieve a specific end product. Sake and soy sauce are good examples. For review, see Pandey et al., Current Science (Bangalore), 77(1):149-163 (1999), herein incorporated by reference. These applications usually involve a high degree of sterilization since humans consume the resulting product. They typically use very thin layers of substrate on trays and involve a lot of material handling to prepare the substrate, grow the appropriate fungus and recover the end product. Generally, food applications justify the cost of production associated with these processes.

In the SSC system provided herein, when a selected fungus is grown on the proper solid nutrient source, it produces a set of enzymes that are functionally different than the enzymes it would produce when grown in a liquid culture. They are more effective in converting cellulose to sugar at ambient temperatures.

In one aspect, the present invention provides process for fungal culture and enzyme preparation employing solid substrate culture. The present invention enables sufficient large scale solid substrate culture.

The present invention provides innovations in physical and biochemical substrate characteristics and process control that reduce costs and improve efficiency of large scale solid state culture. Substrate characteristics induce high product concentrations using low cost materials in large volume cultures, (e.g., up to ten tons of dry weight substrate in a single culture reactor). The present invention also provides methods to control temperature and moisture balance in large scale cultures with very rapid generation of metabolic heat. The selected fungal strain produces multiple cellulase and hemicellulase enzymes when grown in these solid state cultures.

In another aspect, the present invention provides enzyme preparations used in conversion of cellulose to ethanol. The selected fungal strain provide herein is grown in solid state culture to produce an enzyme preparation containing multiple enzyme activities that act on a variety of cellulose and hemicellulose substrates, producing fermentable sugars (for example, glucose and xylose). The enzyme preparation can be used in multiple-step process, where the enzyme preparation is first used to convert cellulose and hemicellulose to sugars, and in a second step the sugars are fermented into ethanol; this is referred to as a separate or “two-step process.” In the separate process the glucose and the five carbon sugars from the hemicellulose hydrolysis may be co-fermented using one yeast strain or fermented separately using different fermentative organisms. Alternatively, the fermentation process step can start before all cellulose is converted into sugar, thus there is some overlap between the cellulose hydrolysis step and the fermentation step. In some embodiments, as described in more detail herein, the enzyme preparation is used in a simultaneous cellulose hydrolysis and fermentation process which combines cellulose, hemicellulose, enzyme, and yeast in a single tank to produce ethanol (referred to herein as a “one-step process.”)

As described below, in some embodiments, the enzyme preparation provided herein comprises the whole solid substrate fungal culture including residual substrate, fungal cells and protein enzymes. When the culture reaches optimal enzyme concentration, the whole culture is harvested. The culture may be used wet without any further processing or may be dried and stored for later use. The culture is a whole culture enzyme preparation containing multiple enzyme activities. The combination of the selected fungal strain and solid substrate culture technology produces sufficiently high enzyme titers that no further processing is required to reach usable enzyme concentrations. This eliminates the principal cost in producing enzymes in conventional liquid fermentation.

Furthermore, exogenous cellulase (for example one or more from different fungus) can also be added to the enzyme preparation.

In another aspect of the present invention, the enzyme preparation provided herein can be further purified, or partially purified, to produce enzymes with higher purity or activities. It can also be used to purify specific enzymes with enzyme purification technologies known in the art.

(1). Substrate Selection and Preparation

The process of producing enzymes by growing a fungus on solid culture begins with selection of the proper fungus and substrate. The selected fungi should be able to metabolize cellulose.

There are known methods of growing fungus on solid substrate; see for example, Ellaiah P. et al. Process Biochemistry, 38(4):615-620 (2002); U.S. Pat. No. 6,558,943.

The present invention provides solid state culture substrates with moisture retention capability and physical strength to use in a packed bed without collapsing or “mushing down”. These solid substrate culture substrates are processed to provide a material with both the physical and nutritional requirements necessary for optimal fungal growth and enzyme production. Some additional soluble nutrients are optionally added to achieve the desired fungal growth and enzyme complex.

Many different solid substrates can be used for the production of enzymes using fungus, such as the production of cellulase employing Trichoderma reesei under solid state fermentation. These include, but are not limited to, wheat straw, wheat bran, corn stalks, switchgrass, wood chips, saw dust, green gram straw, black gram straw, barley straw, oat straw, and rice straw, rice husks, sugar cane bagasse, sugar beet pulp, apple pomice, coffee process waste. In some embodiments, the material used is called BPC (for beeswing, pith and caffe), a fraction of corn cobs from Mount Pulaski Products, Mount Pulaski Ill. This material provides cellulose and hemicellulose as a carbon source, structure to the substrate and water holding capacity. BPC is very water sorbant. Other corn cob fractions such as cob meal which is finely ground whole cob also work. Substrates are generally moistened with water and steam sterilized.

In general, the substrate comprises a mixture of components. The mixture of materials used in the composition of SSC substrate was developed to provide: (1) suitable physical characteristics; (2) nutrition of the fungal growth; and (3) production of the desired mixture of enzyme activities. Substrate ingredients were also selected because they are low cost and readily available in large amounts.

In one aspect, the substrate provided in the present invention comprises a component that provides structural strength and/or moisture reservoir or buffer. Many cellulose containing materials can be used, including but not limited to, corn cob fractions and straw. Corn cob fractions and straw provide physical structure as well as a lost cost means of controlling water activity. In some embodiments corn cob fractions are preferred because of very high water sorbancy and low cost. The corn cob fractions generally absorb three times their weight in water and remain a friable solid. In addition, it was observed that the selected fungal strain grew on cob meal, BPC or straw, but the growth rates were slower and produced low enzyme activity.

In many embodiments, the substrate comprises fractions of corncobs. Corncob is the central wooden core of a maize ear. The majority of a corncob is composed of cellulose (lignocellulose and hemicellulose). Corncob meal, which is obtained by drying and crushing corncobs, is used as a fungal bed for growing mushrooms. Corncobs not only provide a source of cellulase, but also provide both structural strength and a moisture reservoir or buffer. Suitable corncob substrates can be obtained from a variety of sources.

Approximately 60% of the corncob's weight is made up of the hard woody ring. This portion is not a good absorber of water soluble substances. The pith and chaff portion of the corncob are the lighter components that make up the balance of the corncob weight. In their loose form, after having been reduced, for example, by grinding rolls and a hammer mill, these lighter ends can absorb in excess of 350% of their weight in some oils and water and water based liquids. Such a loose, lighter corncob product of chaff and pith which has been separated from the hard woody ring is produced by The Andersons in Maumee, Ohio and is marketed under the trademark SLIKWIK™ (Now owned by Sorbent Products Co. Inc.). BPC (beeswing pith and chaff) is produced by Mount Pulaski Products, Mount Pulaski Ill.

In some embodiments, sugar case bagasse or fractions of bagasse is used to reduce or replace corn cob fractions.

The percentage of corncobs in the total substrate can be from 30 to 80% (w/w), preferably from 45 to 70%, and even more preferably from 45 to 50%, in w/w.

In another aspect, the substrates provided herein for enzyme production comprise a component that provides cellulose as the carbon source, such as straw, corn stover, wood chips, and switchgrass.

Switchgrass (Panicum virgatum) is a warm season grass and is one of the dominant species of the central North American tallgrass prairie. Switch grass can be found in remnant prairies, along roadsides, pastures and as an ornamental plant in gardens. Other common names for this grass include tall panic grass, Wobsqua grass, lowland switch grass, blackbent, tall prairie grass, wild redtop and thatchgrass.

By “straw” herein is meant the dry stalk of a cereal plant after the nutrient grain or seed has been removed. Straw makes up about half of the yield of a cereal crop such as barley, oats, rice, rye or wheat.

In some embodiments grass or grain straw milled to a particle size ranging from 5 mm down to a fine flour is incorporated into the substrate. The percentage of straw may range from 1 to 15% preferably, 3 to 10% and more preferably 5%.

In one aspect, the substrate comprises a component that provides an inducer of cellulase production in the fungus. The biosynthesis of cellulases is induced by cellulose, cellobiose, sophorose and lactose; and repressed by glucose or other readily utilizable carbon sources. The type of inducer that can be used in the present invention depends on the type of fungus that used in the present invention for the production of cellulose. The inducers can be any one known in the art, including, but not limited to, cellulose, lactose, cellobiose, sorbose, cellobionolactone, lactobionic acid, lactulose, and β-glucan, including monosaccharides and disaccharides. It can also be one that is uncovered in an assay for inducer of cellulose production by a given fungus.

A variety of methods can be used for screening for an inducer or test an inducer. For example, to screen for an inducer, a candidate inducer is added to a culture of a cellulase generating microorganism, such as Trichoderma reesei. After continuous cultivation for a suitable period of time (for example, for 96-120 h), cells are separated (for example, by centrifugation at 4° C.) and the obtained supernatant is used for enzyme analyses. The activity of cellulases (FPU) of culture filtrates can be assayed according to the method such as that described by Mandels et al., Measurement of Saccharifying Cellulase. Biochim. Bioeng. Symp., 6:21-23 (1976), and expressed in International Unit (IU), using Whatman No. 1 filter paper. The amount of inducer to be added to the culture can be varied, and preferably a broad range of amount can be used for the screening, such as to achieve a final concentration of 0.1% to 1% (v/v) in the culture. See also, Janas et al., New Inducers For Cellulases Production by Trichoderma Reesei M-7, Electronic Journal of Polish Agricultural Universities, Food Science and Technology, 5(1) (2002) (available online, web address: www. ejpau.media.pl/series/volume5/issue 1/food/art-04.html), herein incorporated by reference.

In some embodiments, the inducer is β-glucan. A glucan molecule is a polysaccharide of D-glucose monomers linked by glycosidic bonds. Some of the commonly known glucans include: cellulose (β-1,4-glucan), laminarin (β-1,3- and β-1,6-glucan), starch (α-1,4- and α-1,6-glucan), glycogen (α-1,4- and α-1,6-glucan), and dextran, (α-1,6-glucan). β-glucans (or beta-glucans) are natural gum polysaccharides occurring in the bran of cereal grains, most abundantly in barley and oats and to a much lesser degree in rye and wheat. In many embodiments of the invention short chain soluble beta 1,3 linked glucans act as inducers of cellulase activities.

The inducer added to the substrate can be in a relative pure form. For example, it can be synthesized chemically, or purified (completely or partial) from a source material. The inducer can also be from a natural source without purification. For example, when β-1,3 glucan is used as an inducer of cellulose production, barley can be used as the source without any purification. Alternatively, an inducer can be provided by genetic engineered microorganisms, such as bacteria, but preferably fungi, that can produce such an inducer. The inducers can be added individually, or in combination to achieve better effects. For example, although lactose alone induces little cellulase under certain conditions, a synergistic effect on cellulase formation was observed following the addition of sophorose, cellobiose or galactose to lactose. Morikawa Y. et al., Cellulase induction by lactose in Trichoderma reesei PC-3-7, Applied Microbiology and Biotechnology, 44(1-2):106-111 (1995), herein incorporated by reference.

Thus, in some embodiments, any material that can provide soluble beta 1,3 linked glucans can be used in the present invention. One such material is barley. Barley (Hordeum vulgare) is a cereal grain, which serves as a major animal feed crop, with smaller amounts used for malting and in health food. It is a member of the grass family Poaceae. In 2005, barley ranked fourth in quantity produced and in area of cultivation of cereal crops in the world (560,000 km²). Barley not only acts as source of nutrition (such as in the form of cellulose and starch), but also may act as an inducer for cellulase and hemicellulase. There is high concentration of beta glucans in barley. Barley may be used in different forms: whole barley ground through a mill to a course or fine powder, barley flour which is barley grin that is dehulled then ground to a fine flour, barley pearling waste which is the hull and a portion of the kernel removed during preparation of pearled barley. In some embodiments, barley and/or wheat germ is added to accelerate fungal growth and induce production of high concentrations of cellulase/hemicellulase activities. In some embodiments, additional inducer, include but is not limited to lactose, is added in addition to barley.

The percentage of barley in the total substrate can be from 10 to 60% (w/w), preferably from 25 to 50%, and even more preferably is about 40%.

In another aspect, substrate provided herein comprises at least one nutrient supplement such as wheat germ. Wheat germ is the vitamin-rich embryo of the wheat kernel. It is generally separated before milling for use as a cereal or food supplement. Wheat germ not only provides as carbon source, but also provides nutrients, such as vitamin and amino acids, which contribute to robust fungal growth.

The percentage of wheat germ in the total substrate can be from 1 to 20% (w/w), preferably from 5 to 15%, and even more preferably is about 10%.

Other additives can also be included, such as soluble nutrients, and minerals can be dissolved in the water used to wet the substrate. Such solutions promote fungal growth but water alone is sufficient for cellulase/hemicellulase production by the fungus. Different kind of salts can be used, such as those generally used as component in culture medium for growing microorganisms, including, but not limited to, ammonium sulfate, potassium phosphate, magnesium chloride, calcium chloride, and trace minerals including ferric chloride, manganese chloride, cobalt chloride, zinc chloride, copper chloride and soluble protein/nitrogen source such as urea, peptone or yeast extract. The nutrient solution added to substrate in the preferred embodiment contains the following in grams per liter: ammonium sulfate 11.6, potassium phosphate 3.8, magnesium chloride 0.3, calcium chloride 0.6, urea 0.6, soy peptone 2.9 and trace minerals each with less than or equal to 0.1 g/liter including ferric chloride, manganese chloride, cobalt chloride, zinc chloride, copper chloride.

Then the components are mixed together, such as by mechanical methods.

In many embodiments, the pH of the substrate is also adjusted to low pH as described herein. The pH can be from 3 to 7, preferably from 4 to 6, and more preferably is about 4.5 to 5.

Many different chemicals can be used to adjust the pH, such ammonia and acid, the later including, but not limited to, sulfuric acid, phosphoric acid acetic acid, lactic acid, citric acid, and hydrochloric acid. The mixing of the substrate and the adjusting of the pH can be carried out in a single step, or in separate steps.

As used herein, the term “about” modifying any amount refers to the variation in that amount encountered in real world conditions of producing sugars and ethanol, e.g., in the lab, pilot plant, or production facility. For example, an amount of an ingredient employed in a mixture when modified by “about” includes the variation and degree of care typically employed in measuring in an ethanol production plant or lab. For example, the amount of a component of a product when modified by “about” includes the variation between batchs in an ethanol production plant or lab and the variation inherent in the analytical method. Whether or not modified by “about,” the amounts include equivalents to those amounts. Any quantity stated herein and modified by “about” can also be employed in the present invention as the amount not modified by “about.”

The substrate components used herein can be processed or raw agriculture products. Many agricultural products, particularly raw products, have indigenous microbial contamination. Left untreated, these contaminants can compete, and potentially out-compete the desired fungi, resulting in a contaminated product, low quality product, or no useable product. As is known in the art, there may be a variety of techniques used to reduce the contamination, including, but is not limited to, heat treatment, steaming, radiation, and treatment with antibiotics.

Steaming finds particular use in the present invention. By “steaming” herein is meant the process of applying vaporized liquid (usually water, although other aqueous solutions are possible) to a material, such as the substrate, for solid state culture described herein. Steaming is one of the common methods of sterilization, for the elimination of microorganisms such as bacteria. Water vaporizes when heated to 100° C. under standard atmosphere pressure (100 kPa). However, under higher pressure, water will only vaporize at temperature higher than 100° C. Thus steaming can be carried out at ambient pressure, such as atmosphere pressure, without extra pressure being applied. Alternatively, steaming can be carried out under pressure higher than 100 kPa (generally referred to as “autoclave”). Autoclaves commonly use steam heated to 121° C. (250° F.), at 103 kPa (15 psi) above atmospheric pressure. Solid surfaces are effectively sterilized when heated this temperature for at least 15 minutes or to 134° C. for a minimum of 3 minutes. “Effective sterilization” in this context means to reduce undesired microorganisms, such as bacteria, to the extent that they cannot interfere with the enzyme production process.

Steaming can also be used to adjust the amount of water in the substrate. A certain amount water is necessary for the growth of the fungus. Water can be added to the substrate together with other components. However, because of the limited amount of water needed for making the substrate, it may be difficult to mix the water evenly in the substrate. Thus, steaming, among others, such as sprinkling during mixing, is a convenient way to introduce water to the substrate evenly. Substrate moisture after steaming may be in the range of 30 to 80% preferably 40 to 50% in barley substrates. Final moisture after addition of a liquid inoculum culture is preferably in the range of 45 to 55%. In some embodiments, water, and or nutrient solution and steam combine to produce final substrate moisture of 40 to 80%, preferably 50 to 70%.

Substrate may be decontaminated by tyndalization or double steaming. Substrate is steamed to about 90° C. cooled, held for 1 hour to 24 hours then steamed a second time to about 90° C. The first steaming kills any vegetative cells and induces spores to germinate and grow. The second steaming kills the vegetative cells from spores that survived the first steaming. Moisture added to the substrate is adjusted so that the final moisture after tyndalization is in the ranges described above. If other decontamination techniques are used, water may need to be introduced separately.

Thus, in one aspect of the present invention, the substrate is steamed to adjust moisture and reduce contamination from indigenous microorganisms. This can be carried in an open space, where the substrate is spread out on a surface, such as the floor, or the bottom of a container. However, preferably, steaming is carried out in a contained space, such as a growth chamber, and optionally, mechanical components are used to move the substrate to assist the dispersion of the steam. Steaming can carried out under pressures higher than atmosphere pressure when steam is introduced into a closed, pressured system. Alternatively, steaming is carried out at ambient pressure, such as the same as the atmosphere pressure, where the steam is introduced into an open system.

The way to generate steam is known in the art, as well as the way of steaming. The duration of the steaming depends on the amount and density of the substrate and temperature and pressure of the steam. It can be from several minutes to several hours, preferably from 5 minutes to one hour, or preferably for 15 to 30 minutes.

After steaming, the substrate will be let cooled down to a temperature suitable for the growth of fungus, either by naturally cooling down over time, or by applying cold air to the substrate.

The substrate then can be used to grow fungus to produce the enzyme preparation of the invention.

In another aspect of the invention, substrate is prepared and microbial contamination reduced by extrusion. Extruders such as those used to produce pasta or pelleted livestock feeds force a material through a small opening in a die where mechanical force creates high temperature and pressure. In the present invention, substrate ingredients are blended, wetted with nutrient solution and extruded through a die to form a pellet. Substrate may be heated in the extruder barrel by steaming or other means to a temperature of 70 to 150 degrees C. prior to being forced through the die. Extruders may be of a single or twin screw design. Temperature in the die ranges from 70 to 200 degrees C. preferably about 150 C and pressure from 100 to 400 psi, preferably about 300 psi. The high pressure and temperature kills contaminating microbes and forms the substrate into a pellet which has good physical characteristics in solid culture.

In one embodiment, the substrate used for the SSC cellulase enzyme preparation is a combination of cellulose containing compounds, primarily a fraction of corn cob and straw supplemented with barley and wheat germ.

In some embodiments the substrate contains 30 to 80% of BPC derived from corn cob, preferably about 45%; Barley flour 10 to 60% preferably 40%; milled grain straw 1 to 5 mm particle size 1 to 15% preferably about 5%, and wheat germ 1 to 20% preferably about 10%. In some embodiments whole ground barley or barley pearling waste may substituted for barley flour. In some embodiments straw may be grass seed or other straw and may be milled to a fine powder up to 10 mm average particle size.

(2). Fungal Inoculum Preparation, Incubation, and Culture Control

The steamed substrate is inoculated with the desired fungus (the inoculum) and loaded into a growth chamber. The fungus grows on the substrate, utilizing it as a food source and at the same time, producing the desired enzymes.

By “inoculum” or “inoculant” herein is meant the material used in an inoculation. For example, the fungus provided in the present invention, or the fungus that are obtained through the methods provided in the present invention, or any other suitable fungus, is produced in conventional liquid culture known in the art to produce a large volume of cell mass. These cells are sprayed on the steamed substrate as an inoculum. In laboratory scale cultures, inoculum is poured onto the prepared substrate and stirred. In larger scale systems, the inoculum is sprayed onto the substrate as the substrate is conveyed into the growth chamber or is sprayed on the substrate as the substrate is mixed. In one embodiment the substrate is steamed in a chamber fitted to allow mixing. After the substrate is steamed and cooled inoculum is sprayed onto the substrate as the substrate is mixed. In another embodiment, substrate pellets from the extruder cool as it is conveyed and inoculum sprayed onto the pellets as pellets are transferred into the growth chamber.

The methods to produce inoculum are well known in the art. Generally, fungus from a stock can be used to grow, either in a liquid medium or on a solid medium, for a period of time under proper temperature. The stock can be obtained from many sources, such as from American Type Culture Collection (ATCC), or other collections of fungus, such as the Fungal Genetics Stock Center at University of Missouri, Kansas City, or a collection kept in-house. The stock can be in the form of conidia (asexual, non-motile spores of a fungus) stored in silica gel or in lyophilized, or as non-spore form kept in a suitable medium for preservation. Any medium can is suitable for the growth of the fungus can be used. The temperature for growing the fungus is 10 to 40° C., preferably 20 to 35° C., and more preferably 30° C. The incubation time is 24 to 96 hours preferably about 48 hours. After the fungus reach the desired density in the liquid culture, or desired colony size on the surface of solid culture media, they are harvested to be used to inoculate substrate in a growth chamber. Either the fungi, the spores formed by the fungi, or mixture of both, can be used as inoculum. Spores can be harvested by methods known in the art, for example, by washing the surface of a agar plate on which fungi grown with either water or buffer, and separate spores by known methods, such as filtering and centrifugation. Spores are easy to store and have a much longer shelf life.

Among the factors that determine morphology and the general course of fungal fermentations, the type and size of inoculum is of prime importance. The preferred embodiment employs a liquid culture inoculum. By “inoculum size” herein is meant the amount of inoculum being used for the inoculation. It is generally measured by the percentage of inoculum weight over the substrate weight. Suitable type and size of inoculum can be determined using methods known in the art. For example, different inoculum size, such as from 0.1 to 20%, can be tested in small scale fermentor to determine the optimal size. Inoculum size can be from 1 to 10%, preferably is about 2-5%.

In one aspect, the present invention provides methods of incubating and growing fungi in a growth chamber or bioreactor using solid state culture technique. The preferred embodiment employs a liquid culture inoculum.

By “growth chamber” or “bioreactor” herein is meant any device or system that supports a biologically active environment, particularly a device capable of holding moist solid fermentation media inoculated with microorganism and carrying out the process of solid state fermentation in a contained manner. A growth chamber can be used to grow any microorganism capable of growing under specified conditions in a contained environment. The bioreactor's environmental conditions like air composition, gas (i.e., air, oxygen, nitrogen, carbon dioxide) flow rates, temperature, pH, humidity, intensity of light, and dissolved oxygen levels, and agitation speed/circulation rate can to be closely monitored and controlled to provide a desired environment for the microorganisms to grow. Bioreactors can be of any size and shape and any configuration that will physically hold the solid substrate in which growth conditions can be maintained. A number of reactor configurations have been tested for solid substrate culture for cellulase enzyme production including columns, cylinders with supporting trays.

In some embodiments, the growth chambers are rectangular in shape and constructed of mild steel or plastic panels designed for ease in cleaning. For example growth chambers designed for commercial use might have dimensions of 10 feet wide, 10 feet high and 60 feet long with a series of trays or shelves stacked at 6 inch to one foot intervals. Shelves are constructed of metal mesh to allow air circulation from the bottom. The growth chamber has doors at both ends of the rectangle. To load the growth chamber inoculated solid substrate is fed onto a flexible net at the “loading end door” as the net is pulled across the support shelf (from the door at the opposite end). When the culture net reaches the length of the growth chamber the net pull is stopped. This is repeated for each shelf. When all shelves are full, the culture incubation proceeds for the desired time. At the conclusion of the culture period the net on each shelf is pulled from the growth chamber at through the “unloading end door”. The fungal culture is scraped from the net onto a conveyor for transport to drying or other processing. The net system allows a small number of people to efficiently load and unload tons of solid substrate culture. Bioreactor makers use vessels, sensors, controllers, and a control system, networked together for their bioreactor system. Fouling (the accumulation and deposition of living organisms and certain non-living material on hard surfaces in an aquatic environment) can harm the overall sterility and efficiency of the bioreactor, especially the heat exchangers. To avoid it the bioreactor preferably is easily cleanable and is as smooth as possible. Biological fermentation is a major source of heat. A flow of temperature controlled air is used to maintain temperature at optimum for fungal growth.

Generally, it is preferred to control the conditions inside the growth chamber throughout the incubation period. The conditions include, but are not limited to, temperature, humidity, pressure, air composition (such as oxygen, carbon dioxide, and nitrogen concentration), pH, and air circulation status. Thus, in many embodiments, the growth chamber preferably is attached to a variety of sensors to monitor the conditions, such as temperature, humidity, pressure, air composition, and pH within the chamber. A variety of sensors are known in the art that can be used to monitor the conditions within the growth chamber. In one embodiment, standard sensors known to the art are used to measure temperature in the substrate and in air, humidity, oxygen concentration in the air, and air flow rate. Thus, during the incubation, one or more parameters can be monitored and/or controlled.

In one embodiment, a Programmable Logic Controller®, PLC®, or Programmable Controller is used to control the reactor. A programmable controller is an electronic device used for automation of industrial processes, such as control of machinery on factory assembly lines. Unlike general-purpose computers, the PLC is designed for multiple inputs and output arrangements, extended temperature ranges, immunity to electrical noise, and resistance to vibration and impact. Programs to control machine operation are typically stored in battery-backed or non-volatile memory. A PLC is a real time system where output result is produced in response to input conditions within a bounded time.

PLC generally has extensive input/output (I/O) arrangements. These connect the PLC to sensors and actuators. PLCs read limit switches, analog process variables (such as temperature and pressure), and the positions of complex positioning systems. On the actuator side, PLCs operate electric motors, pneumatic or hydraulic cylinders, magnetic relays or solenoids, or analog outputs. The input/output arrangements may be built into a simple PLC, or the PLC may have external I/O modules attached to a computer network that plugs into the PLC. PLCs may also have a human-machine interface to interact with people for the purpose of configuration, alarm reporting or everyday control.

In some embodiments, the present invention uses a process control program and PC to monitor pretty simple feedback loops to control Solid Substrate Culture incubation of the fungi. The computer controls electronically actuated valves opened or closed to provide: outside air (or tank gas flow) to responding to temperature measurement and oxygen concentration in chamber atmosphere to control oxygen level; and steam injection into the air flow in response to humidity measurement. Control systems may also divert air flow through heaters or refrigeration to heat or cool the air circulating through the growth chamber.

One property for monitoring and control is temperature. Controlling the temperature of large quantities of rapidly growing fungal culture is preferred in some embodiments. The growth rate of fungus can depend on the temperature. In general, the growth of fungus is a heat generating process, cooling is more likely to be used than heating. If not controlled or removed, metabolic heat generation can increase culture bed temperature to the point where fungal growth is inhibited.

The control of temperature is by transfer of heat in or out of the growth chamber, thus heating or cooling the temperature inside the growth chamber. There are a variety of methods to transfer heat and control the temperature. In some embodiments, the control of the temperature is by circulation of air. For example, circulation of air inside the growth chamber can be coupled with the exchange of the air between the inside and the outside of the chamber. Alternatively, hot or cold air can be blown into the chamber if desired.

In some embodiments, a thermal jacket can be attached to the outside of the chamber, with heat carrying media inside the thermal jacket. The heat carrying media can be solid material or aqueous liquid, such as water, circulation in it. The liquid can be cold or warm, depends on whether cooling or heating is desired. The thermal jacket can be connected to a heating or cooling device. Alternatively, the thermal jacket can comprise a cooling or heating device itself. In some embodiment, the thermal jacket comprises an electric heater. In some embodiments, trays that support culture media may incorporate temperature control by means of circulating a fluid (water) through the tray.

In many embodiments, a constant or substantially constant temperature is maintained inside the growth chamber. This can be accomplished by methods such as agitation of the substrate.

In some embodiment, the transfer of heat between the growth chamber and outside is combined with the agitation of the substrate inside the chamber to maintain a substantially constant temperature inside the growth chamber.

The temperature inside the growth chamber should be controlled for optimal fungus production. It is between from 10 to 50° C., preferably from 20 to 40° C. and even more preferably from 25 to 32° C.

Usually, during the incubation, the fungus metabolizes the substrate, and generates heat (in addition to enzymes). Waste heat is generally low value heat, typically less than 30° C. It can be used as supplemental room heat or exhausted to atmosphere.

The air composition within the chamber is also important. Fungus grows aerobically, thus, sufficient supply of oxygen is important. Carbon dioxide is generated by the fungus, and should be removed from the chamber from time to time to prevent the inhibition of fungus growth. Thus a good air circulation system is provided in many embodiments. Fresh air (for example, from the environment), or gas (in controlled ratio), can be introduced into the chamber to replace the air therein. Generally, air from the atmosphere contains 78% nitrogen, 20.95% oxygen, 0.93% 0.04% carbon dioxide, and about 1% water vapor.

The growth chamber generally also includes at least an inlet and an outlet to allow the circulation of air (or gas) inside the growth chamber. The air come into the chamber is preferably pre-cleaned, such as by filtering, to remove undesired contaminants, particularly bacteria. The source of the air can be from the atmosphere, or from a gas tank with a pre-mixture of gas, including, but not limited to oxygen, nitrogen, and carbon dioxide. Alternatively, gas, such as oxygen, can be pre-mixed with air, and is injected into the growth chamber through an optionally separate inlet. Steam can also be introduced into the growth chamber if desired to maintain the humidity inside the growth chamber. In some case the outlet is connected to a cleaning component, such as a filter, to prevent the released air to contaminate the environment. For example, it can be important to prevent the spores that may be generated during culture from being released from the growth chamber.

The pressure inside the chamber is generally the same as the atmosphere at the site. However, it may be desirable to have pressures lower or higher the atmosphere at the site. For example, the pressure inside the chamber may be lower than outside in order to prevent the spores produced during the incubation from escaping to contaminate the environment. Conversely, the pressure inside the chamber may be higher than outside to prevent microorganisms, such as bacteria, from entering the growth chamber. Generally growth chambers are maintained at positive pressure to prevent introduction of contaminants.

For optimal fungus growth, generally, the oxygen within the chamber is from 15 to 21%, and even more preferably is about 21% as in air. The concentration of carbon dioxide is maintained at normal atmosphere air concentration of about 450 ppm.

The humidity inside the growth chamber is also controlled. Generally, humidity is measured in term of relative humidity (“RH”), which is defined as the ratio of the partial pressure of water vapor in a gaseous mixture of air and water to the saturated vapor pressure of water at a given temperature. During culture incubation, the RH inside the chamber is from 70 to 100% (w/w), preferably from 80 to 100%, and even more preferably from 90 to 95%.

Generally, the substrate pH is adjusted to about pH 5 with addition of mono basic potassium phosphate in the nutrient solution used to wet the substrate. The pH generally is not adjusted during incubation.

The process uses technology innovations that are adapted from the malting and mushroom industries. The mechanics of being able to move large quantities of solid substrate, mix and maintain uniform moisture, and uniformly heat and cool the beds is known in the art. In one aspect, a mixing component is employed to move and mix the substrate within the growth chamber. The mixing component includes, but is not limited to, blades that can blend the substrate, a shaking device on top of which the substrate is placed, a tumbling device, or a device that can move the growth chamber, such as rotating it.

The incubation time varies depending on the enzymes being produced. It is from 3 to 20 days, preferably 5 to 10 days, and more preferably about seven days for growing Trichoderma in solid culture for enzyme production. After the incubation, the whole culture is harvested for next step.

In one embodiment, the fungi metabolize approximately 35% of the substrate during incubation. It exits the growth chamber at 55% moisture. Every 100 lbs of substrate input will result in 65 lbs of enzyme preparation out. On a dry weight basis substrate utilization ranges from about 10% to about 40%, typically about 20%.

(3). Enzyme Preparation

The present invention provides a cellulase/hemicellulase enzyme preparation and methods of making the cellulase/hemicellulase enzyme preparation. The usage of the enzyme preparation as provided in the present invention provides significant cost-reduction in producing ethanol from cellulose.

By “cellulase enzyme preparation” herein is meant the composition containing a mixture of enzymes that efficiently hydrolyze cellulose and hemicellulose under conditions suitable for fermentation. By “cellulase/hemicellulase enzyme preparation” or “SSC cellulase” herein is meant the enzyme mix that comprises cellulase and hemicellulase prepared with the SSC process provided herein. The enzyme activities can be measured by methods known in the art, for example, the filter paper method described herein. The table in the examples shows representative enzyme compositions.

There are commercially available enzyme mixtures for cellulose hydrolysis. For example, Accelase™ 100 enzyme complex (Genencor) contains multiple enzyme activities: exoglucase, endoglucanase, hemi-cellulase and beta-glucosidase. It is produced with a genetically modified strain derived from Trichoderma reesei.

One of the surprising advantages of the SSC cellulase provided herein is that it can be produced with fungus that has not been genetically modified.

In a preferred embodiment, the SSC cellulase is prepared from a fungus that has not been genetically engineered. Thus, the multiple enzyme activities contained in the SSC cellulase are from the process for growing the fungus as provided herein, rather than using a genetic engineered strain of a fungus. In some embodiments, the SSC cellulase is not produced from a genetically modified strain of a Trichoderma reesei. In some embodiments, the strain is genetically engineered.

By “low pH for cellulase” herein is meant the pH from 4 to 7, preferably from 4.5 to 5.0.

By “ambient temperature” herein is meant a temperature between 20 to 40° C. Preferably the temperature is 30 to 35° C.

In many embodiments, the whole culture is used as an enzyme preparation without any purification steps. This way, the cost of producing enzyme preparation is dramatically reduced. Accordingly, the whole culture is slurried and pumped to an ethanol fermentation tank or dried and stored for future use. Since the whole culture is used as the enzyme preparation, there is no significant waste product to dispose of.

If the enzyme preparation is to be used directly in the ethanol production process, water can optionally be added to the whole culture to make a slurry. The amount of water to be added depends on the water content of the whole culture, but is an amount that makes the slurry easily pumped. For example the slurry would be 1 to 10% solids.

The enzyme preparation can be used in the ethanol production methods provided herein or any other suitable process known in the art. For example, it can be used in the process described in U.S. Pat. No. 5,348,871, the entire disclosure of which is incorporated by reference.

Alternatively, the whole culture can be dried for storage using methods known in the art. Cultures are dried using a flow of warm dry air at a temperature of about 20 to 50° C.

In many embodiments, the whole culture is harvested for purifying enzymes that can be used to convert cellulose to sugar. The purification can be carried out according methods known in the art to separate the enzyme proteins from the culture substrate, for example by extracting the culture in water or buffer solution (e.g. ultrafiltration and/or diafiltration), then concentrating the resulting enzyme containing solution or by using known chromatography techniques to purify the enzyme proteins. The purification can be complete or partial.

Generally, the fungi are harvested and separated from the culture media by methods known in the art, such as mixing the culture material in water or buffer solution and centrifugation to separate the liquid fraction containing the enzyme from the residual culture solids. Optionally, the fungi are washed with water or buffer solution, preferably cold, for several times.

To prevent enzyme degradation and denaturing, the purification is preferably carried at low temperature, such as at 4° C., and in the presence proteinase inhibitors. There are many proteinase inhibitors known in the art and are commercially available.

After the enzymes are released from the cell, a variety of methods known in the art can be used for purification. Standard purification methods include chromatographic techniques, such as ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation (such as ammonium sulfate precipitation), dialysis, and chromatofocusing techniques. Ultrafiltration and diafiltration techniques, in conjunction with protein concentration, are also useful.

The amount of SSC Cellulase enzyme preparation required for cellulose hydrolysis depends on the enzyme activities of the enzyme preparation as well as the nature of the feed stock that provides the source of the cellulose. Thus, the enzyme activities of the enzyme preparation and the content of the cellulose of the feedstock can be measured using methods known in the art or those described herein. Small scale pilot runs can also be conducted to determine the optimal amount of enzyme preparation needed.

In some embodiments, the SSC Cellulase enzyme preparation is typically added to the ethanol fermentation at about 0.5 to 30% % w/w whole culture enzyme preparation to feedstock. In the SSC system, data expresses enzyme loading as weight percent of dry whole SSC culture to dry feedstock input. Because the process design generally employs whole culture material without any separation or purification, enzyme preparations contain cell mass and residual substrate including protein in substrate components. The whole crude enzyme preparation is used on a weight basis in the hydrolysis process.

The enzyme activities contained in the preparations provided herein are defined by selective substrate enzyme assays and found to generally include endo and exo acting cellulases, cellobiase, and xylanases. Hydrolysis of cellulose was determined in assays measuring the conversion of filter paper to glucose. Optimal cellulose hydrolysis activity is at pH 4.5 to 5.0. Cellulose hydrolysis occurs at 20 to 50° C. Enzyme preparations produced by the methods provided in the present invention were compared to commercial cellulase preparations. The SSC produced cellulases contained less filter paper units per gram than did the commercial cellulases. However in assays comparing hydrolysis of delignified straw, SSC cellulases were superior to commercial cellulases on an equal weight basis.

In one aspect, the enzyme composition provided by the present invention comprises cellulases. By “cellulase (E.C.3.2.1.4)” herein is meant enzymes that catalyze the cellulolysis (or hydrolysis of cellulose). Cellulases are produced chiefly by fungi, bacteria, and protozoans. There are also cellulases produced by other types of organisms such as plants and animals. Several different kinds of cellulases are known, which differ structurally and mechanistically. There are five general types of cellulases based on the type of reaction catalyzed. They are endo-cellulases, exo-cellulases, cellobiases, oxidative cellulases, and cellulose phosphorylases.

Thus in one aspect, the enzyme preparation comprises endo-cellulase. Endo-cellulase breaks internal bonds to disrupt the crystalline structure of cellulose and expose individual cellulose polysaccharide chains.

In one aspect, the enzyme preparation comprises exo-cellulase. Exo-cellulase cleaves 2-4 units from the ends of the exposed chains produced by endocellulase, resulting in the tetrasaccharides or disaccharide such as cellobiose. There are two main types of exo-cellulases (or cellobiohydrolases): one type working processively from the reducing end, and one type working processively from the non-reducing end of cellulose.

In another aspect, the enzyme preparation comprises cellobiase. Cellobiase (or beta-glucosidase) hydrolyzes the endo-cellulase product into individual monosaccharides.

Methods for measuring the activities of endo-cellulase, exo-cellulase, and cellobiase are known in the art. See e.g., Howard G. T. and Elliot L. P., Effects of Cellulolytic Ruminal Bacteria and of Cell Extracts on Germination of Euonymus americanus L. Seeds, Applied and Environmental Microbiology, 54(1):218-224 (1988), herein incorporated by reference.

In yet another aspect, the enzyme preparation comprises hemicellulase. In some embodiments, the hemicellulase is xylanse. Xylanase is a class of enzymes that degrade the linear polysaccharide beta-1,4-xylan into xylose, thus breaking down hemicellulose, which is a major component of the cell wall of plants.

Methods for measuring the activities of endo-cellulase, exo-cellulase, and cellobiase are known in the art. See e.g., EP 1433844, herein incorporated by reference.

Additional enzymes may be added in the compositions and methods encompassed by the invention. For example, cellobiose, an intermediate disaccharide formed according to equation (3), inhibits the hydrolysis reaction. Therefore, if there is an insufficient amount of cellobiase in the enzyme preparation, extra amount of cellobiase can be added to increase the hydrolysis efficiency.

In some embodiments, extra amounts of endo-cellulase, exo-cellulase, cellobiase and/or xylanses can be added to the enzyme preparation for better conversion of cellulose to sugar. These enzymes can be obtained commercially from Genencor, logen, and Novozyme, or through methods known in the art. For example, high concentration cellulase/xylanase complex and beta glucanase/xylanase complex are marketed by Genencor International Inc.

The effective amount of these enzymes to be included in the methods of the invention can be readily determined by one skilled in the art.

### C. Cellulose Hydrolysis

Generally, in biomass-to-biofuels (B2B) processes, after harvest, biomass is reduced in size and then treated to dissociate the lignin from the cellulose and hemicellulose in a step that can take from a few minutes to many hours. Several methods can be used for this purpose, such as biomass treatment with saturated steam at 200° C., explosion with ammonia, and cooking with warm dilute acid. Dilute acid pretreatments are fast (minutes), whereas steam-based treatments can take up to a day. After pretreatment, the solid suspension is exposed to cellulolytic enzymes that digest the cellulosic and hemicellulosic biomass components to release the hydrolysis products, primarily six- and five-carbon sugars, respectively (along with acetic acid and lignin-derived phenolic by-products). The type of pretreatment defines the optimal enzyme mixture to be used and the composition of the hydrolysis products. The latter are fermented by ethanol-producing microorganisms, naturally occurred, selected, or genetically engineered, including but not limited to, yeasts, Zymomonas mobilis, Escherichia coli, or Pichia stipitis.

The integrated cellulosic, starch feedstock process provided in the present invention can use any source of cellulosic lignocellulosic material (such as wood or wood waste, switch grass, etc.) as an energy feedstock.

By “cellulosic biomass” or “cellulosic feedstock” herein is meant materials that contain cellulose. It includes, but is not limited to, wood or wood waste, straw, herbaceous crops, corn stover, grass such as switch grass, or other sources of annual or perennial grass, or any delignified cellulose such as paper or paper waste, pulp and paper mill waste, municipal and industrial solid wastes.

The cellulosic feedstock primarily consists of cellulose, hemicellulose, and lignin bound together in a complex structure along with small quantities of extractives, pectins, proteins, and ash. Due to the complex chemical structure of the cellulosic feedstock, microorganisms and enzymes cannot effectively attack the cellulose without prior treatment because the cellulose is highly inaccessible to enzymes or bacteria. This inaccessibility is illustrated by the inability of cattle to digest wood with its high lignin content even though they can digest cellulose from such material as grass. Successful commercial use of biomass as a chemical feedstock depends on the separation of cellulose from other constituents.

In general, as described below, cellulosic feedstock is grounded or processed to reduce the particle size and/or increase surface/volume ratio.

In the first step of the process, the lignocellulosic material is ground and pretreated to separate the lignin from the cellulose. The pretreatment step can employ any one of a number of processes to separate lignin including: dilute acid, steam explosion, ammonia explosion, solvent extraction, high temperature/high pressure oxygen or alkaline. The pretreatment process can either recover cellulose only or may be a process that recovers both cellulose and hemicellulose. The hemicellulose may either be recovered as the polymer or hydrolyzed to constituent sugars by the pretreatment process.

By “cellulose” as used herein, is meant a polysaccharide of beta-glucose that has the formula (C₆H₁₀O₅)ₙ. Cellulose forms the primary structural component of green plants. The primary cell wall of green plants is made of cellulose; the secondary wall contains cellulose with variable amounts of lignin. By “hemicellulose” herein is meant a mixture of polysaccharides composed primarily of polymers of xylose, arabinose and galactose.

Cellulosic feedstock in an untreated or minimally treated form is referred to as “raw feedstock”; treatments as outlined herein results in the “cellulosic feedstock” in the saccharification process.

The amount of cellulose can be measured by methods known in the art. For example, Updegraff D M, Semimicro Determination of Cellulose in Biological Materials, Analytical Biochemistry 32: 420-424 (1969) herein incorporated by reference.

Many cellulosic feedstocks contain lignin. Lignin and cellulose, considered together, are termed lignocellulose; lignocellulosic feedstocks can also be used in the present invention. It is noted that cellulosic feedstocks and lignocellulosic feedstocks are not mutual exclusive terms. One of the abundant lignocellulosic feedstock is sugarcane residue, called bagasse, generated during the milling of sugarcane and is plentiful in tropical and subtropical regions. Other lignocellulosic feedstocks include, but are not limited to, agricultural residues such as corn stover, wheat and rice straw, and forestry residue; industrial residue such as pulp and paper processing waste; and energy crops such as switchgrass. Unlike starch which contains homogenous and easily hydrolyzed polymers, lignocellulose plant matter contains cellulose (23-53%), hemicellulose (20-35%), polyphenolic lignin (10-25%) and other extractable components. Knauf M., and Monirussaman M., Ligocellulosic biomass processing: A perspective, International Sugar Journal, 106(1263):147-150 (2004), herein incorporated by reference.

Different methods known in the art can be used to remove lignin from the cellulosic material. In some embodiments for lignocellulosic feedstocks, the present invention employs an alkaline pretreatment process that recovers both cellulose and hemicellulose. This step is not necessary if already delignified cellulose such as paper or pulp and paper mill waste is used in the process.

In the second step, cellulose and hemicellulose are hydrolyzed to the constituent sugars, glucose from cellulose and mixed sugars including xylose and arabinose from hemicellulose. Hydrolysis can be accomplished by acid or enzymatic processes employing any cellulase, hemicellulase preparations. Cellulose, hemicellulose hydrolysis can be a separate process step preceding fermentation or can be combined in a simultaneous hydrolysis and fermentation. Pulp and paper mill waste, waste paper or other delignified cellulose may not contain a hemicellulose fraction and require only cellulose hydrolysis.

### II. Fermentation of Hydrolyzed Cellulose Material

In another aspect, the present invention provides processes of fermentation. With the addition of yeast or ethanol producing bacteria, fermentation may take place as a separate unit operation. Alternatively the steps of hydrolysis and fermentation may be combined by adding yeast or bacteria together with enzymes in a simultaneous hydrolysis and fermentation. In either approach, the fermentation organism may be one such as the yeast Saccharomyces cerevisiae that only ferments glucose; may be an organism that ferments five and six carbon sugars derived from hemicellulose; or may be an organism that co-ferments glucose and hemicellulose derived sugars. The organism may also be one that both produces cellulase enzymes and ferments sugars to ethanol. Depending on the pretreatment process and process flow of hemicellulose, and on the choice of fermentation organism, the fermentation may follow one of multiple process options: A) The glucose derived from cellulose hydrolysis may be fermented and hemicellulose or hemicellulose derived sugars left unfermented; B) The glucose derived from cellulose hydrolysis and the sugars derived from hemicellulose hydrolysis may be co-fermented in a single step using one organism; C) The glucose and hemicellulose derived sugars may be fermented in separate, parallel or sequential steps using different organisms. Regardless of the fermentation process, the final ethanol concentration in any fermentation stream will preferably be less than 7% ethanol and typically in the range of 2.5 to 5% w/w ethanol.

In some embodiments, the present invention employs a yeast strain that ferments both glucose and hemicellulose derived sugars in a batch, in a simultaneous hydrolysis and fermentation with a final ethanol concentration of 2.5% to 5% w/w.

In some embodiments, the present invention employs enzymatic hydrolysis using a mixed activity preparation containing cellulose and hemicellulose hydrolyzing enzymes in a simultaneous hydrolysis and fermentation. This step is typically conducted at pH 4.5 to 5.0 and at a temperature of 30° C. to 50° C.

Alternatively, the process may employ the SSC cellulase enzyme preparation described herein or other enzyme preparations containing only cellulase activities to hydrolyze delignified cellulose feedstocks.

In some embodiments, the process comprises simultaneous cellulose hydrolysis. In some embodiments, the process runs via a batch or continuous process.

A. Simultaneous Cellulose Hydrolysis and Fermentation

The present process can include simultaneous saccharification and fermentation, using reagents and conditions described above for saccharifying and fermenting. The simultaneous hydrolysis and fermentation generates less heat than a straight fermentation process where all the glucose is made prior to the addition of yeast, thus reduces cooling costs. Generally, in simultaneous hydrolysis and fermentation, yeast consume sugars as they are produced, limiting feedback inhibition of the enzymes. Preferably, the enzyme preparation provided in the present invention is used in the process of simultaneous hydrolysis and fermentation of cellulose described in more details below.

In order for nearly complete fermentation, and in order to produce large quantities of ethanol, the common practice has been to use a batch process wherein extremely large fermentation vessels capable of holding upwards of 500,000 gallons are used. With such large vessels, it is economically unrealistic to provide an amount of yeast sufficient to rapidly ferment the sugar solution. Hence, conventional fermentation processes have required 72 hours and more because such time periods are required for the yeast population to build to the necessary concentration. For example, a quantity of yeast is added to the fermentation vessel. In approximately 45-60 minutes, the yeast population will have doubled; in another 45-60 minutes that new yeast population will have doubled. It takes many hours of such propagation to produce the quantity of yeast necessary to ferment such a large quantity of sugar solution.

The present invention provides an ethanol production process—simultaneous hydrolysis and fermentation of cellulose using SSC cellulase. In this ethanol production process SSC cellulase enzyme and yeast are combined with the mash in a single step in one fermentation vessel with fermentation at fermentation temperature and low pH.

In some embodiments, saccharification and fermentation is conducted at a pH of about 6 or less, and preferably about 4.5 to 5.0. The initial pH of the saccharification and fermentation mixture can be adjusted as described herein. In one embodiment, saccharification and fermentation is conducted for about 24 hours to 7 days, preferably about 48 to 96 hours or more preferably about 72 hours. In an embodiment, the temperature can be decreased as ethanol is produced. For example, in an embodiment, during fermentation the temperature can be as high as about 37° C. and then reduced to about 25° C. This temperature reduction can be coordinated with increased ethanol titers (%) in the fermentor. The preferred temperature range is the maximum tolerated by the yeast, generally about 35° C. (but below 45° C. which is the optimum for the enzyme.)

In one embodiment, simultaneous saccharifying and fermenting can be carried out employing quantities of enzyme preparation and yeast selected for effective fermentation without added exogenous nitrogen; without added protease; and/or without added backset. Backset can be added, if desired, to consume process waters and reduce the amount of wastewater produced by the process.

The amount of enzyme of preparation can be adjusted as to generate optimal output. For example, simultaneous saccharifying and fermenting can employ enzyme preparation at about 1 to 30 and preferably 2.5 to 10% (w/w), of dry solids cellulosic material.

The saccharification and/or fermentation mixture can include additional ingredients to increase the effectiveness of the process. For example, the mixture can include added nutrients (e.g., yeast micronutrients), antibiotics, salts, added enzymes, and the like. Nutrients can be derived from stillage or backset added to the liquid. Suitable salts can include zinc or magnesium salts, such as zinc sulfate, magnesium sulfate, and the like. Extra enzymes can be added, such as protease, phytase, cellulase, hemicellulase, exo- and endo-glucanase, xylanase, and the like.

The concentration of cellulose in the mash and ratio of enzyme to cellulose determines the final concentration rate of ethanol production. Biomass can be up to 40% solids, final ethanol concentration up to 14% v/v and total hydrolysis fermentation time from about 36 to 72 hours. The time is a function of the enzyme and cellulose concentrations. Generally, in practice maximum solids content is limited by viscosity to about 10 to 15% and the maximum ethanol concentration about 3 to at most 7%

The product of the fermentation process is referred to herein as “beer”. For example, fermenting corn produces “corn beer”. Ethanol can be recovered from the fermentation mixture, from the beer, by any of a variety of known processes. For example, ethanol can be recovered by distillation. The remaining stillage includes both liquid and solid material. The liquid and solid can be separated by, for example, centrifugation. The recovered liquid, thin stillage, can be employed as at least part of the liquid for forming the saccharification and fermentation mixture for subsequent batches or runs.

In one embodiment, pretreated cellulose feedstock, enzyme preparation, yeast and water are combined in a single step in one fermentation vessel at approximately ambient temperature. A 30° C. operating temperature helps optimize the performance of the yeast, but the process can operate at lower temperatures. A pH of 4.5 is used to help prevent microbial contamination. The simultaneous hydrolysis and fermentation occur in 24-96 hours, depending on the concentration of cellulose in the slurry, size of the substrate particles and the amount of enzyme added to the mash. The process flow is shown in following diagram in FIG. 2.

The present invention provides the use of SSC cellulase, which is cost effective, in commercial production systems for producing ethanol from cellulose. The SSC cellulase provided by the present invention can be used on waste cellulosic materials that contain no lignin (e.g., waste paper) or low amounts of lignin (Oregon grass straw, switch grass, barley straw, etc.).

B. Continuous Fermentation

The SSC Cellulase provided herein can be used in a batch or continuous process. A continuous process includes moving (pumping) the saccharifying and/or fermenting mixtures through a series of vessels (e.g., tanks) to provide a sufficient duration for the process. For example, a multiple stage fermentation system can be employed for a continuous process with 48-96 hours residence time. For example, cellulosic material (e.g., fractionated plant material) can be fed into the top of a first vessel for saccharifying and fermenting. Partially incubated and fermented mixture can then be drawn out of the bottom of the first vessel and fed in to the top of a second vessel, and so on.

Without being bound by theory, it is believed that the present method is more suitable than conventional methods for running as a continuous process. The present process should provide reduced opportunity for growth of contaminating organisms in a continuous process. At present, the majority of dry grind ethanol facilities employ batch fermentation technology. This is in part due to the difficulty of preventing losses due to contamination in these conventional processes. For efficient continuous fermentation using traditional technology, the conventional belief is that a separate saccharification stage prior to fermentation is necessary to pre-saccharify the cellulose for fermentation. Such pre-saccharification insures that there is adequate fermentable glucose for the continuous fermentation process.

A continuous stirred tank reactor (CSTR) process overcomes at least some of the limitations of batch processes. The CSTR process features continuous stirring or agitation of the substrate slurry by, for example, mechanical mixing or liquid recycling. The CSTR process allows optimization and balancing of the hydrolysis and fermentation rates to eliminate the large accumulation of glucose and the resulting inhibition of ethanol production. The CSTR process employs continuous addition of fermentable substrate, catalysts and fermentation agents, and continuous removal of any residual substrate—and product—containing broth. The CSTR process has perpetually high concentrations of microorganisms, much reduced down time compared to batch reactors, generally lower maximum concentrations of potentially inhibitory mono- and disaccharides, but higher ethanol concentration. Thus, the relative merits of batch and CSTR will depend upon the needs and circumstances surrounding a given application.

The use of a continuous solids retaining bioreactor (CSRB) provides further improvements in the production of ethanol. The CSRB improves productivity and yield by providing differential solids retention and thus increasing the concentration of substrate particles in the reactor and increasing the hydrolysis rate. The use of a CSRB increases the overall hydrolysis rate and thus reactor productivity by maximizing the amount of cellulose/enzyme complex in the reactor. The key to efficiency in the CSRB process appears to be the management and control of the cellulose/enzyme complex in the reactor.

A further advancement in the production of ethanol is the use of cascaded CSRBs, in which the output from one CSRB reactor vessel becomes the input feed to the next CSRB reactor vessel. This arrangement overcomes the problem of decreased or limited productivity enhancement with high conversion, as the cascaded reactors achieve higher total conversion for an equal cumulative residence time. However, the solids retention in the later stages is generally less than in the early stages as a result of reduced cellulose particle size, because smaller particles require more time to settle. An advantage of the cascaded CSRB system over the single CSRB is that at high conversion, the presence of large amounts of ethanol in a single CSRB inhibits the further production of ethanol, whereas this inhibition is alleviated to some extent in a cascade system because the average concentration of alcohol seen by the reaction is reduced as the reaction proceeds through sequential steady state reactors at increasing ethanol concentration until the final concentration is reached.

U.S. Patent Application Publication No. 20060014260 is also incorporated herein by reference. It discloses a semi-continuous simultaneous saccharification and fermentation (SSF) process for the bioconversion of cellulose into ethanol and other organic chemicals

### III. Hydrolysis of Starch

In one aspect, the present invention provides a process that integrates cellulose process streams with a “no cook” or reduced temperature starch hydrolysis process, as described in more detail herein.

Conventional industrial ethanol plants employ amylase (starch degrading) enzymes in a multi-step process. First, a slurry or “mash” is made and then heated to 80° C. to 120° C. to hydrate and gelatinize the starch granules. In grain (or any natural source of plant starch), starch is contained in insoluble granules. This cooking step is necessary in the convention process to hydrate the starch granules to make the starch accessible to the enzymes.

By “mash” as used herein, is meant as a mixture of a fermentable carbon source (carbohydrate) and water used to produce a fermented product, such as an alcohol. Specifically, it refers to a mixture of hot water and crushed grain, which also can be used to produce malt beverages. In some embodiments, the term “beer” and “mash” are used interchangeably.

The mash is then cooled to a lower temperature, typically about 98° C., and an alpha amylase enzyme is added to break the starch polymer into short chains of glucose. It is further cooled to 35° C. to 55° C. and then glucoamylase is added to produce individual glucose molecules. The temperature at which the enzymes are added is dependent on the heat tolerance of the enzymes used. It is cooled again to approximately 30° C. and yeast is added to convert the glucose to ethanol (fermentation). Fermentation of the sugars generates metabolic heat which is removed from the process. After fermentation, the fermentation mixture called beer typically contains approximately 12% ethanol. This mixture (beer) is distilled to concentrate the ethanol. The non-starch portion of the grain is carried through the entire process and is recovered after distillation to make distiller dry grains (or DDGs).

Any starch hydrolysis and fermentation methods known in art can be used in the present invention, as long as they are compatible with the spirit of the present invention.

In some embodiments, amylase from commercial sources, such as Novozyme and Genencor, can be used. For example, any amylase that can hydrolyze starch at a temperature lower than the boiling temperature of ethanol can be used the in the present invention.

In some embodiments, the fermentation beer from the cellulose ethanol production is integrated with the “no cook” starch hydrolysis process, also known as Ambient Temperature Starch Hydrolysis (“ATSH”), a process for ethanol production from plant material. Preferably, the ATSH is carried out using the enzyme produced by the Solid State Culture (SSC) technology described herein.

In bioethanol production, enzymes are responsible for converting starch to sugars. The sugars are then fermented with yeast to make a beer containing 2-15% ethanol. One major problem in the traditional fermentation process is the contamination of undesired microorganisms. The “cooking” step in the conventional process allows both the killing of contaminating organisms and can serve as a type of pre-processing step of the starch, to “open up” the starch granules for better access to the starch enzymes. However, in some cases, additional methods for decontamination may be necessary. Current methods used to kill these unwanted microorganisms, among others, often involve introduction of foreign agents, such as antibiotics, heat, and strong chemical disinfectants, to the fermentation before or during production of ethanol. Commonly, synthetic chemical antibiotics are added to the fermentation vessels in an attempt to decrease the growth of lactic acid producing bacteria. The addition of each of these foreign agents to the process significantly adds to the time and costs of ethanol production. The use of heat requires substantial energy to heat the fermentation vessels as well as possibly requiring the use of special, pressure-rated vessels that can withstand the high temperatures and pressures generated in such heat sterilizing processes. Chemical treatments can also add to the cost of production due primarily to the cost of the chemicals themselves, and in addition these chemicals are often hazardous materials requiring special handling and environmental and safety precautions.

The present invention provides one alternative cost effective solution—to conduct the fermentation process under low pH, which will limit the growth of the contamination microorganisms, as well as the optional added benefit of the use of ambient temperature, which can significantly reduce the cost of the process.

Accordingly, the invention provides a strain of Aspergillus phoenicis (deposited with the USDA Agricultural Research Service Culture Collection, Peoria, Ill., U.S.A as NRRL-50090), although as is outlined herein, other strains may be screened for use in the present invention. The strain is grown on a solid state substrate (sometimes referred to as “solid culture substrate” or “solid fermentation substrate” as outlined below), which optionally and preferably includes barley to induce the production of enzymes. The growth of the fungus on the substrate results in an enzyme composition that produces a variety of enzymes, optionally including amylase, glucoamylase and beta-glucanase, that have activity at low pH and ambient temperature. This is significant as the enzymatic activity at low pH allows the enzyme composition to be used on starch mashes that have undergone the traditional “cooking” step, instead relying on low pH to prevent the growth of indigenous and unwanted organisms during the starch processing.

In one aspect, the present invention provides growth substrates and growing conditions that allow production of enzyme preparations using fungus, such as the strain of Aspergillus provided herein. The invention can be generally described as follows. The substrate is selected to provide nutrition for fungal growth and the physical structure of the solid substrate culture. The dry substrate is moistened with added water or a nutrient containing solution, then steamed to adjust moisture and reduce contamination from indigenous microorganisms. The steamed substrate is cooled and inoculated with the desired fungus and loaded into a solid support growth chamber. The final moisture content of the substrate is such that the moisture is absorbed into the substrate and the substrate remains solid. The fungus grows on the substrate, utilizing it as a nutrient source, and at the same time producing the desired enzymes. This incubation time varies depending on the enzymes being produced. After the incubation, the whole culture is harvested to obtain the enzyme preparation. In many embodiments, the whole culture is used for converting starch to sugar and no additional purification of the enzymes is required. Alternatively the enzymes can be extracted and purified from the culture substrate. These enzyme preparations can be used in a process called Ambient Temperature Starch Hydrolysis (“ATSH”) that also provided herein to convert uncooked starch into sugar at ambient temperature and low pH.

Thus, the present invention provides enzyme preparations used in conversion of starch to ethanol. The selected fungal strain provided herein is grown in solid state culture to produce an enzyme preparation containing multiple enzyme activities that act on a variety of starch substrates, including raw ungelatinized starch granules, producing fermentable sugars (glucose and soluble short chain glucose polymers). The enzyme preparation can be used in multiple-step processes, where the enzyme preparation is first used to convert starch to sugar, and in a second step where the sugar is fermented into ethanol; this is referred to as a “two-step process.” Alternatively, the fermentation process step can start before all starch is converted into sugar, thus there is some overlap between the starch hydrolysis step and the fermentation step. In some embodiments, as described in more detail herein, the enzyme preparation is used in a simultaneous raw starch hydrolysis and fermentation process which combines raw, ungelatinized (“uncooked”) grain mash, enzyme, and yeast in a single tank to produce ethanol.

This enzyme composition can then be added to starch mashes for starch hydrolysis, and used in conjunction with a yeast to produce ethanol.

Thus, the starch processing parts of the present invention provides two steps: an enzyme production step, and then a secondary ethanol production step optionally conducted without heating the mash to gelatinization temperatures and at low pH. Eliminating the cooking step reduces capital cost, operating cost and process energy in ethanol production.

### Solid Substrate Culture Technology

The instant application further provides solid substrate culture technology (sometimes referred to as solid state fermentation) to produce enzyme preparations capable of converting starch to glucose (sugar) at ambient temperatures. The use of these enzyme preparations allows production of sugar from starch without the traditional “cooking” step used in most enzymatic sugar production. Eliminating the cooking step saves capital cost and energy.

Conventional ethanol processes uses enzymes produced in liquid fermentation. Solid substrate culture is different from conventional liquid fermentation. In a liquid fermentation system, a microorganism is placed in a liquid environment that contains soluble nutrients. Air or oxygen is bubbled through the liquid using agitation or injection to dissolve oxygen in the liquid. Generally there is not any solid support media and the oxygen available to the organism is limited by the solubility of oxygen in water. In general, these are specific enzymes that are concentrated and frequently purified to some extent

Furthermore, when more than one enzyme is used in an ethanol process (e.g., alpha amylase and glucoamylase used in hydrolysis of starch to glucose), the individual enzymes are generally produced from different organisms grown in separate liquid fermentation vessels. One of the primary costs of enzymes produced in liquid culture is the cost of concentrating the enzymes or separating the enzymes from the broth in which they are grown. The more liquid in the process, the higher the transportation, storage, and purification costs. The present invention provides new methods of producing enzymes at high concentrations, and generally, does not require post-production purification.

Accordingly, the instant invention provides a solid substrate culture technology that results in enzyme preparations produced from one organism with high enzyme concentrations that contains all of the enzyme activities necessary to work effectively in downstream ethanol production including from raw, uncooked starch. It should be noted that while the description herein is generally directed to processes that are run at lower pH and ambient temperatures, the enzyme preparations (or enzymes purified and/or concentrated from the enzyme preparations) also find use in traditional starch processes, or as individual enzymes for use in a wide variety of applications as is known in the art.

In the SSC substrate for the ATSH enzyme process described herein, water content varies from about 40 to 60% w/w depending on the actual substrate, producing a moist solid particle mix with no free water. The organism obtains oxygen from the air or from modified atmosphere introduced into the growth chamber, as is more fully described below.

### A. Substrate Selection and Preparation

The process of producing enzymes by growing a fungus on solid culture begins with selection of the proper fungus and substrate. The selected fungi should be able to metabolize starch.

In general, the substrate comprises a mixture of components.

In one embodiment, a component of the substrate used for the ATSH amylase production is barley. Barley (Hordeum vulgare) is a cereal grain, which serves as a major animal feed crop, with smaller amounts used for malting and in health food. Barley not only acts as source of nutrition for the fungus, but also appears to act as an inducer for amylase production. Experimental evidence suggests that short chain soluble beta 1,3 linked glucans act as very powerful inducers of amylase activities.

In one embodiment, the substrate comprises barley, primarily steam rolled or hulled barley. To form the substrate, one embodiment utilizes steam rolled or hulled barley, which is wetted with a nutrient solution and steamed prior to inoculation as described below. Other forms of barley may be used as well

In another embodiment, finely ground barley is mixed with a nutrient solution (at between 20 and 60% moisture) and extruded to form pellets which comprise the solid culture substrate. The extrusion process creates high temperature and pressure as the barley/water is forced through the extruder die so that steaming the substrate to control contamination is not necessary.

By “rolling” or “grinding” herein is meant the processes used to reduce the size of whole grains. The physical forces employed include, but is not limited to, impact to create fractures, abrasion/attrition to scrape off material, shear to slice apart, and pressure to crush (deform) structure. Particle size reduction is important to solid state culture for the following reasons. First, particle size reduction increases surface area, which leads to improved utilization of grains through increased exposure of endosperm material to fungus. Second, particle size reduction provides improved mixing characteristics of dissimilar substrate ingredients. Third, particle size reduction provides improved handling of fibrous feedstuffs.

By “steamed rolled” herein is meant the process in which steam is applied to barley before rolling. Steam rolled barley is produced by exposing barley to steam for three to five minutes and then rolling it. This process produces fewer fines than dry rolling or grinding. Steamed rolled barley is commonly used as feedstock in the cattle industry to increase feed consumption and weight gain.

By “hulled” herein is meant the outer hull is removed from barley. The separation of outer hull from inner barley groat can be done by methods of centrifugal force. Barley grains can be gravity fed on to the center of a horizontally spinning stone to be thrown to the out ring where the oat and hull will separate from to the impact. The lighter barley hulls will be then aspirated away while the denser barley groats will be taken to the next step of processing.

The extent of rolling can be controlled. Typically the rolled barley can be used in the present invention is from commercial feed mill and is just a flattened barley kernel maybe 2 to 4 mm thick. Finely ground barley used to make the extruded pellets is barley ground in a hammer mill or other mill to produce a powder or flour that passes a 20 mesh US standard screen. The barley may be whole grain or may be hulled prior to milling.

The percentage of barley in the total substrate can be from 10 to 99% (w/w), preferably from 50 to 90%, and even more preferably from 80 to 90%. Typically dry steam rolled or dehulled and steam rolled barley is mixed with water/nutrient solution to about 40 to 50% moisture content, (equal weight of barley and solution gives a 50% moisture content). The solid substrate can also include straw. The function of the straw is to open up the culture bed structure to facilitate aeration. In some embodiments, the substrates comprise straw in pieces of 0.5 to 3 cm long at rate of about 1 to 5% w/w on a dry basis.

As will be appreciated by those in the art, many different combinations of substrates can be used including those outlined herein for fermentation. The description herein is meant to include all possible combinations of substrates, including combinations that lack particular components.

In addition to the grain components, the invention utilizes a wetting solution. In one embodiment, this solution can be water, which is sufficient for fungal growth and enzyme production on substrates, including barley substrates, although in general, higher titers of enzymes are produced when a nutrient wetting solution is used. Thus the term “wetting solution” includes both water as well as solutions containing additional nutrients and/or chemicals such as acid to control pH.

Thus, nutrient wetting solutions find use in the present invention in many applications. The nutrient solution can add additional nutrients in a variety of forms for use by the fungus, as well as be used to adjust the pH of the substrate. For example, nutrient solutions can contain nitrogen sources, acids and bases or buffers, and minerals. Nutrient wetting solutions of particular use include solutions containing urea, ammonium phosphate and sulfuric acid. A particular nutrient solution contains urea at 16 grams per liter, ammonium phosphate at 13.3 grams per liter, and sulfuric acid at 13.3 ml one molar solution per liter of water. In some embodiments, stillage is added as nutrient wetting solution as described herein. In some embodiments molasses solution of 1 to 10% is added as a nutrient wetting solution.

In general, the wetting solution is added to the substrate to result in a desired final moisture content, although in some cases additional water or nutrient solution can be added periodically to the fungal fermentation as well. For example, if steaming of the substrate is used to reduce bacterial contamination is used, as is described below, this step generally introduces additional water. Thus, the final moisture content of the substrate, as well as the moisture content that is maintained during the enzyme production, can be reached using nutrient solutions and/or water.

The substrate components used herein can be processed or raw agriculture products. Raw agricultural products frequently have indigenous microbial contamination. Left untreated, these contaminants will compete with the slower growing fungus, and potentially out-compete the desired fungi, resulting in a contaminated product, low quality product, or no useable product. As is known in the art, there may be a variety of techniques used to reduce the contamination, including, but is not limited to, heating (including steaming), radiation, and treatment with antibiotics. In some embodiments, a steaming process is employed to handle large quantities of solid materials.

Steaming finds particularly use in the present invention. By “steaming” herein is meant the process that applying vaporized water to a material, such as the substrate for solid state culture described herein. Steaming is one of the common methods of sterilization for the elimination of microorganisms such as bacteria. Water vaporizes when heated to 100° C. under standard atmosphere pressure (100 kPa). However, under higher pressure, water will only vaporize at temperature higher than 100° C. Thus steaming can be carried out at ambient pressure, such as atmosphere pressure, without extra pressure being applied. Alternatively, steaming can be carried out under pressure higher than 100 kPa. Steaming carried out under pressure higher than 100 kPa is called autoclaving. Autoclaves commonly use steam heated to 121° C. (250° F.), at 103 kPa (15 psi) above atmospheric pressure. Solid surfaces are effectively sterilized when heated this temperature for at least 15 minutes or to 134° C. for a minimum of 3 minutes. “Effective sterilization” in this context includes methods to reduce undesired microorganisms, such as bacteria, to the extent that they can not interfere with the enzyme production process.

With the Aspergillus amylase solid substrate culture process described herein, steaming at ambient pressure is sufficient; that is, pressure sterilization of the substrate is not necessary (although it can find use in some processes).

By “ambient pressure” herein is meant a pressure that is close to the atmosphere pressure in a given site. The atmosphere pressure changes according to the altitude and latitude, and can be measured by standard atmosphere (1 atmosphere=101.325 kPa). Thus for most locations, the ambient temperature is about 100 kPa.

By “ambient temperature” here is meant a temperature that is between 15-50° C., and preferably is between 18-40° C., and more preferably the temperature is 35° C.

By “Standard ambient pressure and temperature” herein is meant 25° C., 100 kPa.

Steaming can also optionally be used to adjust the amount of water in the substrate. A certain amount of water is necessary for the growth of the fungus. Water can be added to the substrate together with other components. However, because there is only a limited amount of water needed for making the substrate, it may be difficult to mix the water evenly in the substrate. Thus, steaming, among other techniques, such as sprinkling during mixing, is a convenient way to introduce water to the substrate evenly. Substrate moisture after steaming may be in the range of 30 to 80% preferably 40 to 50% in barley substrates.

If other decontamination techniques are used, water may need to be introduced separately. If the substrate is extruded, for example using barley pellets, ground barley is mixed with water or nutrient solution to a 30 to 60% moisture content to form a moist solid dough which is forced through the extruder die at any combination of pressure and temperature sufficient to form a moist solid pellet. The preferred pressure may range from 50 to 300 psi and temperature from 50 to 150° C. Any equipment capable of forming extruded pellets by forcing material through a die may be employed.

Thus, in one aspect of the present invention, the substrate is steamed to adjust moisture and reduce contamination from indigenous microorganisms. In one embodiment, the substrate is steamed by applying steam. This can be carried in open space, where the substrate is spread out on a surface, such as the floor, or the bottom of a container. However, preferably, steaming is carried out in a contained space, such as in a growth chamber, and optionally, mechanical methods are used to mix and move the substrate to assist in the even distribution of steam throughout the substrate. Steaming can carried out under pressure higher than atmospheric pressure when steam is introduced into a closed, pressured system. Alternatively, steaming is carried out at ambient pressure, such as the same as the atmosphere pressure, where the steam is introduced into an open system.

The duration of the steaming depends on the amount and density of the substrate. It can be from several minutes to several hours, preferably from 10 to 30 minutes up to 4 hours. Substrate may also be double steamed in a process called tyndalization. In this process the substrate is steamed for a period preferably 1 to 30 minutes, then allowed to cool to about 30° C. and held for a period of 4 to 24 hours, preferably about 12 hours. The substrate is then steamed again for a period of 10 to 30 minutes.

After steaming, the substrate will be let cooled down to a temperature suitable for the growth of fungus, either by naturally cooling down over time, or by applying cold air to the substrate.

Final moisture after addition of a liquid inoculum culture is preferably in the range of 45 to 55%. Final moisture content of the substrate is determined by the absorbency or water holding capacity of different substrate materials under different process conditions of temperature a pressure. With barley substrates final moisture content can range from the minimum water activity at which the selected fungal strain will grow, about 30% moisture in barley substrates, to a maximum at which the substrate is no longer solid, in barley substrates about 80% final moisture. Final moisture refers to moisture content of the substrate after nutrient solution or water addition, steaming and inoculation. Then the components are mixed together, preferably by a mechanical method.

Optionally, the pH of the substrate is also adjusted to low pH. The pH can be from 3 to 7, preferably from 3.5 to 5. As is known in the art, many different chemicals can be used to adjust the pH, such acid, including, but is not limited to, ammonia, sulfuric acid, phosphoric acid acetic acid, lactic acid, citric acid, and hydrochloric acid. The mixing of the substrate and the adjusting of the pH can be carried out in a single step, or in separate steps.

As used herein, the term “about” modifying any amount refers to the variation in that amount encountered in real world conditions of producing sugars and ethanol, e.g., in the lab, pilot plant, or production facility. For example, an amount of an ingredient employed in a mixture when modified by “about” includes the variation and degree of care typically employed in measuring in an ethanol production plant or lab. For example, the amount of a component of a product when modified by “about” includes the variation between batches in an ethanol production plant or lab and the variation inherent in the analytical method. Whether or not modified by “about,” the amounts include equivalents to those amounts. Any quantity stated herein and modified by “about” can also be employed in the present invention as the amount not modified by “about.”

The substrate then can be used to grow fungus.

### B. Fungal Inoculum Preparation, Incubation and Culture Control

The methods to produce inoculum are well known in the art. Generally, fungus from a stock can be used to grow on a medium, either liquid or solid, for a period of time under proper temperature. The temperature is 20-45° C., preferably 20° C.-35° C., and more preferably 30° C. The incubation time is one day to one month, preferably 2 to 20 days, more preferably 2 to 15 days, and even more preferably 1 to 5 days. After the fungus reach the desired density in the liquid culture, or desired colony size on the surface of solid culture media, they are harvested to be used to inoculate substrate in a growth chamber. Either the fungal cells, the spores formed by the fungi, or mixture of both, can be used as inoculum. Spores can be harvested by methods known in the art, for example, by washing the surface of an agar plate or on a solid culture substrate of smaller volume than the production culture on which fungi grown with either water or buffer, and separate spores by known methods, such as filtering and centrifugation. Spores are easy to store and have a much longer shelf life.

Among the factors that determine morphology and the general course of fungal fermentations, the type and size of inoculum is of prime importance. For example, different inoculum size, such as from 0.1-20% (w/w), can be tested in small scale fermentor to determine the optimal size. Inoculum size can be from 0.1-20%, preferably from 0.5 to 5%, and even more preferably from 1-2%, and even more favorably is about 2% (w/w). In one embodiment, the selected fungal strain is grown on a solid culture substrate such as the barley substrate described above until the fungus produces spores. The spore culture is then dried and stored. The spore culture material is used to inoculate the solid culture substrate such that the ratio of spores to substrate is in the range of 100 to 100 million spores per gram of substrate, preferably about one million spores per gram of substrate. As will be appreciated in the art, the size of the inoculum can range depending on the desired time of growth.

The fungus provided herein can be grown in a liquid medium know in the art. In some embodiments, the A. phoenicis is grown in a liquid media consisting of 5% molasses and 1% ammonium phosphate. In other embodiments, A. phoenicis is grown in a YM broth, a standard laboratory media containing yeast extract, malt extract and glucose.

In solid culture, the fungus grows on the surface of and penetrates into the moist solid substrate particles. Fungal cells are directly exposed to atmospheric oxygen. Dissolved oxygen and the aeration agitation necessary in liquid culture is generally not relevant to the solid culture system. In solid culture systems reported in the literature temperature is controlled by air flow through the culture substrate and or by mechanical systems such as temperature controlled trays or heat exchangers in the culture bed. In some embodiments, air flow and air temperature are used as one method of temperature control. However a very important innovation in the system of the present invention is manipulation of the gas composition of the atmosphere to control the rate of metabolism and of metabolic heat generation. Specifically the present invention allows cultures to deplete oxygen and or enrich the atmosphere with carbon dioxide or nitrogen in response to culture temperature. This slows metabolic rate and reduces heat generation during periods of peak metabolic rate in the culture.

Specific designs for solid substrate culture equipments are described, for example, in U.S. Pat. Nos. 6,197,573, 6,664,095, and 6,620,614, herein all incorporated by reference.

Controlling the temperature of large quantities of rapidly growing fungal culture is preferred in some embodiments. If not controlled or removed, metabolic heat generation will increase culture bed temperature to the point where fungal growth is inhibited.

The present invention provides processes to both control metabolic rates and efficiently remove heat while maintaining substrate moisture. The packed substrate bed is designed to allow air circulation and heat removal. Control of bed moisture and air humidity is an important factor in the success of solid substrate systems. Air circulation will tend to dry the substrate, which can reduce the amount of water below the point where fungi will grow, unless additional wetting solution is added. Temperature control for the Aspergillus SSC is also key, as the A. phonencis strain grows very rapidly in certain systems, which can generate very high peak heat loads.

Thus, the present invention includes controlling the metabolic rate of growth of the fungus. A suitable control process employed with Aspergillus cultures monitors and controls the oxygen content in the culture atmosphere to control the metabolic rate of the fungus. By using carbon dioxide from the substrate metabolism supplemented by controlled additions of either nitrogen or carbon dioxide to the atmosphere in the SSC chamber the present invention provides a process that can control the rate of metabolic heat generation and manage peak heat loads in the culture without affecting the titer or composition of the enzyme complex. Typically oxygen concentration in the culture is monitored and controlled between about 2% and about 5% to reduce peak metabolic heat generation and prevent peak metabolic heat generation from increasing culture substrate temperature above 36° C. At about 5% oxygen concentration metabolic heat generation is slowed sufficiently to prevent a rise in culture temperature. At the 2% oxygen, metabolic rate is generally slowed sufficiently to reduce culture temperature if necessary. In addition, maintaining oxygen concentration at 5% or less prevents spore formation by the Aspergillus strain (or by other fungal strains tested). Preventing spore formation brings multiple advantages to SSC: exposure to spores may cause allergic response in sensitive individuals; airborne spores are difficult to control and may contaminate other cultures or processes conducted in a facility; and spore color in final enzyme preparations might cause problems with customer acceptance. While the presence or absence of spores makes no difference to the composition of final enzyme preparation, users may find the black color imparted by the Aspergillus spores undesirable; without spores the whole culture enzyme preparations are a brownish color common in the industry.

In some embodiments, for optimal fungus growth at the beginning of the growth period, the growth chamber atmosphere is maintained with normal fresh air with 20.1% oxygen and about 0.04% carbon dioxide. To control metabolic heat generation and aid in temperature control, introduction of fresh air is limited to allow oxygen to be depleted and CO₂ to increase. In addition nitrogen or CO₂ may be introduced into the air circulation to reduce oxygen concentration. During peak metabolic period, oxygen is maintained between 2% and 5% to reduce metabolic heat generation and aid in temperature control. Fresh air may be introduced again to maintain culture growth.

Fungi can secrete metabolites that may change the pH of the substrate during the course of incubation. Thus, it may necessary to adjust the pH during the course of incubation. Generally, the pH inside the growth chamber is 3-6, preferably 3-5, and more preferably is about 3.5. Acid is added to the substrate along with other nutrients to reduce the pH to about 4. Generally, pH is not adjusted during culture incubation

The process uses technology innovations adapted from the malting and mushroom industries. The mechanics of being able to move large quantities of solid substrate, mix and maintain uniform moisture, and uniformly heat and cool the beds is known in the art. In one aspect, a mechanical method is employed to move and mix the substrate within the growth chamber. The mechanical methods can be blades that can blend the substrate, or a shaking device on top of which the substrate is placed. Any mechanical system to efficiently mix a solid material such as barley substrate with water solutions is suitable for the present invention. In one system, dry substrate in a “mixing chamber” is stirred by the action of hollow flight agars set vertically that lift the substrate up through the center of the agar where it falls out the top providing vertical mixing. While turning, the agars travel horizontally through the substrate to mix the entire substrate bed. Water nutrient solution and or steam may be added while the substrate mixes. Alternatively substrate can be mixed and wetted using paddle mixers of standard commercial design. Water, nutrient solution or steam can be added to the substrate during operation of the paddle mixer. After wetting and steaming, substrates (for example composed of steam rolled barley or barley flakes) can be inoculated and transferred into the growth chamber using conveyor systems. In another system, finely ground barley mixed with a water solution in a paddle mixer or other mixing device is fed through an extruder to form substrate pellets. The pellets are inoculated and loaded into the growth chamber. The growth chamber also includes at least a gas inlet and a gas outlet to allow the circulation of the air inside the growth chamber. The air introduced into the chamber is preferably pre-cleaned, such as by filtering, to remove undesired contaminants, particularly bacteria. The source of the air can be from atmosphere, or from a gas tank. The air can be mixed with other gases in the growth chamber including but not limited to oxygen, nitrogen, and carbon dioxide, all of which may be generated on-site. Alternatively, gas, such as oxygen, nitrogen and carbon dioxide can be injected separately, pre-mixed with air or each other, and injected into the growth chamber through a separate inlet. Steam can also be introduced into the growth chamber if desired to maintain the humidity inside the growth chamber. The outlet is optionally connected to a cleaning method, such as a filter or scrubber, to prevent the spores in the released air from reaching the environment. Such spores, especially spores from a fast growing fungus, may contaminate other facilities nearby and affect the production.

The growth chamber preferably is also attached to a variety of sensors to monitor the conditions, such as temperature, humidity, pressure, air composition, and pH within the chamber. A variety of sensors are known in the art and can be used to monitor the conditions with the growth chamber.

The growth chamber is also preferably attached to control methods that can control the conditions, such as temperature, humidity, pressure, air composition, within the chamber.

The temperature inside the chamber is generally warmer than the atmosphere at the site. However, it may be desirable to have growth chamber pressure lower or higher than the atmosphere at the site. For example, the pressure inside the chamber may be lower than outside in order to prevent the spores produced during the incubation from escaping to contaminate the environment. Conversely, the pressure inside the chamber may be higher than outside to prevent microorganisms, such as bacteria, from entering the growth chamber. Growth chambers usually operate under positive pressure relative to the outside atmosphere.

For optimal fungus growth, generally, the oxygen within the chamber is from about 1 to about 30%, preferably from about 2 to about 21%, and during periods of peak metabolism from 2 to 5%. Generally, the oxygen concentration starts of at atmospheric concentrations, it could be higher but that may add an unnecessary cost and doesn't necessarily increase metabolism. The oxygen concentration can be anything above 5% without inhibiting growth and/or enzyme production for the first 12-18 hours. After 12 hours the oxygen concentration generally drops to 5-9%. After 18 hours (the onset of peak metabolism), the oxygen concentration generally drops to 2-5%. The fungus will grow at lower oxygen (less than 1%) but the growth becomes inconsistent through the bed, affecting the consistency of enzyme production. At oxygen concentrations above 5%, it is generally necessary to employ mechanical means to remove heat, i.e. refrigeration, agitation, etc. The concentration of carbon dioxide and/or nitrogen will increase proportionally as the oxygen concentration decreases. The concentration of carbon dioxide is from 0.04 to 98% (w/w), preferably from 0.4 to 19%, and even more preferably during and after peak metabolism from 17 to 19% w/w.

The humidity inside the growth chamber is also controlled. The RH inside the chamber is from 10 to 100 (w/w), preferably from 50 to 95%, and even more preferably from 90 to 95%. Generally, the relative humidity will be whatever atmosphere RH is at the time the chamber is loaded (e.g., typically 10-15% in Montana). As soon as it is loaded, the chamber is closed and the humidity level is raised as high as possible (95% is the general limit on measuring humidity) and maintained as high as possible during the entire growth cycle. This is done to help prevent the substrate from drying out.

The incubation time varies depending on the enzymes being produced. It is from 2 to 15 days, preferably 3 to 7 days, and more preferably about four days for growing Aspergillus for ATSH. After the incubation, the whole culture is harvested for the next step in the process.

In one embodiment, the fungi metabolize approximately 45% to 55% of the substrate during incubation. It can exit the growth chamber at about 50% moisture. On a dry basis, for each 100 lbs of substrate input, 45 to 55 lbs of enzyme preparation is recovered.

### C. Enzyme Preparation

There are commercially available purified enzymes for starch hydrolysis. For example, alpha-amylase and glucoamylase preparations are marketed by Genencor International Inc. (SPEZYME® series of thermostable alpha-amylase and DISTILLASE® series of glucoamylase) and by Novozymes Inc.

The present invention provides a raw starch enzyme preparation and methods of making the enzyme preparation. The usage of the enzyme preparation as provided in the present invention provides significant cost-reduction in producing ethanol from starch.

By “starch enzyme preparation” herein is meant the composition containing a mixture of enzymes that efficiently hydrolyze starch in raw ungelatinized starch granules at low pH and ambient temperature.

By “low pH for amylase” herein is meant the pH from 3.5 to 5.5, preferably from 3.5 to 4.5.

In many embodiments, the whole culture is used as an enzyme preparation without any purification steps. This way, the cost of producing enzyme preparation can be dramatically reduced. Accordingly, the whole culture is mixed in water and pumped to an ethanol fermentation tank or dried and stored for future use. Since the whole culture is used as the enzyme preparation, there is no waste product to dispose of.

If the enzyme preparation is to be used directly in the ethanol production process, water can be added to the whole culture to make a slurry. The amount of water to be added depends on the characteristics of the pump. Typically the slurry will contain 30%-50% w/w solids.

The enzyme preparation can be used in the ethanol production methods provided herein or any other suitable process known in the art.

Alternatively, the whole culture can be dried for storage using methods known in the art. The whole culture can be air dried at temperatures below 36° C., a freeze drier can be used, or a vacuum dryer.

in some embodiments, the whole culture is harvested for purifying enzymes that can be used to convert starch to sugar. The purification can be carried out according methods known in the art to separate the enzyme proteins from the culture substrate, for example by extracting the culture in water or buffer solution, then concentrating the resulting enzyme containing solution or by using known chromatography techniques to purify the enzyme proteins. The purification can be complete or partial, and can include just removing the remaining solids and fungal cells or higher levels of purification as outlined herein, including diafiltration, ultrafiltration, and chromatography.

Generally, the fungi are harvested and separated from the culture media by methods known in the art, such as centrifugation. Generally, secreted amylases are recovered from the liquid cultures as follows: The culture supernatant was adjusted to 20% saturated ammonium sulfate and stirred for one hr at 4° C. After centrifugation, the resultant supernatant was adjusted to 70% saturated ammonium sulfate and stirred for one hr at 4° C. After centrifugation of the supernatant, the resultant pellet was re-dissolved in 50 mM sodium acetate, pH 6.0, 5 mM calcium chloride, and sterile filtered. When SSC is used, enzymes can be recovered by washing the culture with a cold buffer (such as PBS) before being adjusted to 20% saturated ammonium sulfate as above.

In another embodiment, the whole culture is used as an enzyme preparation without any purification steps. This way, the cost of producing enzyme preparation is dramatically reduced. Accordingly, the whole culture is slurried and pumped to an ethanol fermentation tank or dried and stored for future use. Since the whole culture is used as the enzyme preparation, there is no waste product to dispose of.

If the enzyme preparation is to be used directly in the ethanol production process, water can be added to the whole culture to make a slurry. The amount of water to be added depend on the type of pump used to transfer the slurry to the fermentation tank. Typically the slurry would contain about 30-50% dry weight whole culture solids.

Alternatively, the whole culture can dried for storage using methods known in the art. The whole culture can be dried by any method in which the temperature of the culture substrate does not exceed 36° C. during the drying process. In some embodiments, warm dry air is circulated around and through the substrate. During initial stages of drying, air temperature may exceed 50° C. as evaporative cooling maintains substrate temperature below 36° C. As the culture dries air temperature is reduced.

The amount of ATSH enzyme preparation required for starch hydrolysis depends on the enzyme activities of the enzyme preparation as well as the nature of the feed stock that provide the source of the starch. Thus, the enzyme activities of the enzyme preparation and the content of the starch of the feedstock can be measured using methods known in the art or those described herein. Small scale pilot runs can also be conducted to determine the optimal amount of enzyme preparation needed. Enzyme activities are determined as discussed below.

In one embodiment, the ATSH enzyme preparation is typically added to the ethanol fermentation on the basis of a ratio of total weight of dry weight equivalent whole culture to the dry weight equivalent of the ethanol substrate. This ratio may be in the range of 0.125% to 20% of whole culture to ethanol feedstock or 1.25 to 200 grams dry weight whole culture to each 1000 grams of whole ground starch containing ethanol feedstock. The preferred process for ethanol production from raw starch uses a simultaneous starch hydrolysis and fermentation in which ground grain (or other starch containing ethanol feedstock) is mixed with water to form a mash, acid added to adjust pH and to which enzyme and yeast are added such that the glucose from the enzymatic hydrolysis of the raw starch is immediately converted to ethanol by the yeast. In this process the overall rate of ethanol production is determined by the rate of raw starch hydrolysis which is determined by the ratio of enzyme to raw starch—the rate of raw starch hydrolysis. In some embodiments enzyme to feedstock ratio is such that the complete conversion of starch to ethanol takes place over a time period with a final ethanol concentration typical of the conventional fermentation process. This is generally in the range of 0.25% to 5% enzyme to ethanol feedstock to achieve a final ethanol concentration in the fermented “beer” of 10 to 15% ethanol in 36 to 72 hours.

One assay used to assess total raw starch hydrolytic activity in whole culture enzyme preparations was a standardized simultaneous hydrolysis and fermentation. This assay is described in more details in the Examples. This assay was the most useful for assessing efficiency of raw starch activity as it is a measure of overall conversion of raw grain starch to ethanol. Other assays were used to evaluate enzyme preparations but no one assay predicted efficiency of raw starch conversion. Other assays used are described in the Examples and below.

The enzyme activities contained in the preparations were defined by selective substrate enzyme assays as described above and found to include alpha-amylases, glucoamylases, debranching (alpha 1,6 linkage) enzymes and beta glucanases. As noted herein, enzyme preparations that require no further purification find particular use in the invention. Hydrolysis of raw, granular starch is determined in assays measuring glucose and total soluble sugars from raw starch granules and by observing pitting and disappearance of raw starch in microscope examination. Optimal raw starch hydrolysis activity is at pH 3.5 to 3.8. Raw starch hydrolysis occurs at 10 to 50° C. It is not necessary to heat the starch to elevated temperatures to just below the gelatinization temperature of starch. The enzyme preparation hydrolyzes raw starch from any grain, grain waste material (for example residual material from manufacture of pearled barley) potatoes, or any other starch containing material. The beta glucanase content is a particular advantage with barley feedstock. These enzymes hydrolyze the beta glucans contained in barley, which create high mash viscosity in barley mash that increases capital and operating costs.

One component of the enzyme composition is alpha-amylase. By “α-amylase (e.g., E.C. class 3.2.1.1)” herein is meant enzymes that catalyze the hydrolysis of alpha-1,4-glucosidic linkages (thus also known as 1,4-α-D-glucan glucanohydrolase; glycogenase). These enzymes have also been described as those effecting the exo or endohydrolysis of 1,4-α-D-glucosidic linkages in polysaccharides containing 1,4-α-linked D-glucose units. By acting at random locations along the starch chain, α-amylase breaks down long-chain carbohydrates, ultimately yielding maltotriose and maltose from amylose, or maltose, glucose and “limit dextrin” from amylopectin. Because it can act anywhere on the substrate, α-amylase tends to be faster acting than β-amylase. Another term used to describe these enzymes is “glycogenase”. Exemplary enzymes include alpha-1,4-glucan 4-glucanohydrase glucanohydrolase.

The alpha-amylase of the invention is characterized by its ability to hydrolyze carbohydrates under acidic conditions. An amylase produced by fungi and able to hydrolyze carbohydrates under acidic conditions is referred to herein as acid fungal amylase, and is also known as an acid stable fungal alpha-amylase. Acid fungal amylase can catalyze the hydrolysis of partially hydrolyzed starch and large oligosaccharides to sugars such as glucose. The acid fungal amylase that can be employed in the present process can be characterized by its ability to aid the hydrolysis of raw or native starch, enhancing the saccharification provided by glucoamylase

Alpha amylase activity may be measured by using the DNS method as described in Miller, G. L. (1959) Anal. Chem. 31:426 428, U.S. Patent Application Publication No. 20030125534, and Food And Nutrition Board, National Research Council, Food Chemicals Codex (5th ed. 2003) (hereinafter Food Chemicals Codex) herein all incorporated by reference. The amount of acid fungal amylase employed in the present process can vary according to the enzymatic activity of the enzyme preparation. In general, activities of 40 to 70 alpha amylase units as defined by the assay method described in Example 4 were used. Other assays that can be used are the soluble substrate assay and starch hydrolysis assay described in U.S. Pat. No. 5,736,499, herein is incorporated by reference in its entirety. Additional assays are described in more detail in the Examples.

The enzyme composition provided in the present invention also optionally comprises “glucoamylase.” By “glucoamylase” herein is meant the amyloglucosidase class of enzymes (e.g., EC.3.2.1.3, glucoamylase, 1,4-alpha-O-glucan glucohydrolase), an enzyme that removes successive glucose units from the non-reducing ends of starch. These are exo-acting enzymes, which release glucosyl residues from the non-reducing ends of amylose and amylopectin molecules. The enzyme also hydrolyzes alpha-1,6 and alpha-1,3 linkages although at much slower rate than alpha-1,4 linkages. Glucoamylases are produced by several filamentous fungi and yeasts, with those from Aspergillus being commercially most important.

Glucoamylase activity may be assayed by the 3,5-dinitrosalicylic acid (DNS) method. Goto et al., Biosci. Biotechnol. Biochem. 58:49-54 (1994). The amount of glucoamylase employed in the present process can vary according to the enzymatic activity of the enzyme preparation. In general, activities of 300 to 500 glucoamylase units as defined by the assay method described in Example 4 were used. Additional assays are described in more detail in the Examples.

The enzyme composition provided herein may also include beta-glucanases. By “beta-glucanase” herein is meant the enzyme that can digest beta glucan, such as the beta glucan from barley.

Method for assaying beta-glucanase can also be found in Food Chemicals Codex and Walsh et al., Journal of Animal Science, 73(4):1074-1076 (1995), herein are incorporated by reference. Additional assays are described in more detail in the Examples.

Additional enzymes may be added in the compositions and methods encompassed by the invention.

In some embodiments, extra enzymes may be added to the enzyme preparations of the present invention. These enzymes include both carbohydrases as well as additional enzymes.

In some embodiments, for example, additional carbohydrases can be added, for example, additional α-amylase(s) can be added. Fungal amylase can be isolated from any of a variety of fungal species, including Aspergillus, Rhizopus, Mucor, Candida, Coriolus, Endothia, Enthomophtora, Irpex, Penicillium, Scierotium and Torulopsis species. In an embodiment, the acid fungal amylase is thermally stable and is isolated from Asperguillus species, such as A. niger, A. saitoi or A. oryzae, from Mucor species such as M. pusillus or M. miehei, or from Endothia species such as E. parasitica. In an embodiment, the acid fungal amylase is isolated from Aspergillus niger. In addition, many of these fungal enzymes, including α-amylase, can be purchased and added to the processes of the invention; see for example, SPEZYME® series of thermostable alpha-amylase and DISTILLASE® series of glucoamylase from Genencor International Inc., and Spirizyme® brands of glucoamylase and Termamyl® brands of alpha-amylase by Novozymes Inc.

Another carbohydrase enzyme that may be added to the compositions of the invention are beta-amylases (E.C. 3.2.1.2). These are exo-acting maltogenic amylases, which catalyze the hydrolysis of 1,4-alpha-glucosidic linkages in amylose, amylopectin and related glucose polymers.

Additional carbohydrase enzymes include but are not limited to debranching enzymes such as pullulanases (E.C. 3.2.1.41) and isoamylases (E.C. 3.2.1.68). Such enzymes hydrolyze alpha-1,6-glucosidic bonds. Thus, during the hydrolysis of the starch, debranching enzymes remove successive glucose units from the non-reducing ends of the starch.

Further additional enzymes which may be used are proteases, such as fungal and bacterial proteases. Fungal proteases include for example, those obtained from Aspergillus, Mucor and Rhizopus, such as A. niger, A. awamori, A. oryzae and M. miehei. Other enzymes include but are not limited to cellulases, hemicellulases, lipases cutinases, and lignase.

The effective amount of these enzymes to be included in the methods of the invention can be readily determined by one skilled in the art.

In some embodiments, an antimicrobial may be added to the compositions and fermentation medium of the invention. Antimicrobials are compounds that kill or inhibit the growth of microorganisms.

### D. Ambient Temperature Starch Hydrolysis

The present invention provides Ambient Temperature Starch Hydrolysis (“ATSH”) processes for ethanol production from plant material.

The starting plant material is generally processed to produce a mash that has starch in a form more accessible and thus more easily converted than the starting plant material. By “plant material” herein is meant all or part of any plant (e.g., cereal grain), typically a material including starch. Suitable plant material can be any starch containing material, includes grains such as maize (corn, e.g., whole ground corn, either standard corn or waxy corn), sorghum (milo), waxy or standard barley, wheat, rye, rice, triticale and millet; and starchy root crops, tubers, or roots such as potato, sweet potato and cassava.

The present method converts starch from plant material (e.g., fractionated plant material) to ethanol. The plant material (e.g., fractionated plant material) can be reduced by a variety of methods, e.g., by grinding, to make the starch available for saccharification and fermentation. Other methods of plant material reduction are available. For example, vegetable material, such as kernels of corn, can be ground with a ball mill, a roller mill, a hammer mill, or another mill known for grinding vegetable material, and/or other materials for the purposes of particle size reduction. Also can be used is emulsion technology, rotary pulsation, sonication, magnetostriction, ferromagnetic materials, or the like. These methods of plant material reduction can be employed for substrate pretreatment. Although not limiting to the present invention, it is believed that these methods can increase surface area of plant material (e.g., fractionated plant material) while raising the effectiveness of flowing of liquefied media (i.e. decreased viscosity). These methods can include electrical to mechanical, mechanical to electrical, pulse, and sound based vibrations at varying speeds. This can provide varying frequencies over a wide range of frequencies, which can be effective for pretreating the plant material (e.g., fractionated plant material) and/or reducing particle size.

Although not limiting to the present invention, it is believed that certain of these sonic methods create low pressure around a particle of plant material (e.g., fractionated plant material) and induce cavitation of the particle or disruption of the particle structure. The cavitated or disrupted particle can increase availability of plant material (e.g., starch) to an enzyme, for example, by increasing surface area. It is believed that such pretreatment can decrease quantity of enzyme rates in the present method for ethanol production.

In one embodiment, the present method includes vibrating plant material (e.g., fractionated plant material) and cavitating the fluid containing the plant material. This can result in disrupting the plant material and/or decreasing the size of the plant material (e.g., fractionated plant material). In certain embodiments, the present method includes treating plant material (e.g., fractionated plant material) with emulsion technology, with rotary pulsation, with magnetostriction, or with ferromagnetic materials. This can result in disrupting the plant material and/or decreasing the size of the plant material (e.g., fractionated plant material). In another embodiment, the present method includes sonicating the plant material (e.g., fractionated plant material). This can result in disrupting the plant material and/or decreasing the size of the plant material (e.g., fractionated plant material).

In one embodiment, the present method can include employing sound waves for reducing plant material (e.g., fractionated plant material). The sound waves can be ultrasound. The present method can include sonicating the plant material (e.g., fractionated plant material). The method can include sonicating the plant material at a frequency (e.g., measured in kHz), power (e.g., measured in watts), and for a time effective to reduce (or to assist in reducing) the particle size to sizes described hereinabove. For example, the method can include sonicating the plant material (e.g., fractionated plant material) at 20,000 Hz and up to about 3000 W for a sufficient time and at a suitable temperature. Such sonicating can be carried out with commercially available apparatus, such as high powered ultrasonics available from ETREMA (Ames, Iowa).

In one embodiment, the present method can include employing rotary pulsation for reducing plant material (e.g., fractionated plant material). The method can include rotary pulsating the plant material (e.g., fractionated plant material) at a frequency (e.g., measured in Hz), power (e.g., measured in watts), and for a time effective to reduce (or to assist in reducing) the particle size to sizes described hereinabove. Such rotary pulsating can be carried out with known apparatus, such as apparatus described in U.S. Pat. No. 6,648,500, herein is incorporated by reference.

In an embodiment, the present method can include employing pulse wave technology for reducing plant material (e.g., fractionated plant material). The method can include rotary pulsing the plant material at a frequency (e.g., measured in Hz), power (e.g., measured in watts), and for a time effective to reduce (or to assist in reducing) the particle size to sizes described hereinabove. Such pulsing can be carried out with known apparatus, such as apparatus described in U.S. Pat. No. 6,726,133, herein is incorporated by reference.

A fine grind exposes more surface area of the plant material, or vegetable material, and can facilitate saccharification and fermentation. In one embodiment, the vegetable material is ground so that a substantial portion, e.g., a majority, of the ground material passes a sieve with a 0.1-0.5 mm screen. In another embodiment, about 35% or more of the ground vegetable material can fit through a sieve with a 0.1-0.5 mm screen. In yet another embodiment, about 35 to about 70% of the ground vegetable material can fit through a sieve with a 0.1-0.5 mm screen. In an embodiment, about 50% or more of the ground vegetable material can fit through a sieve with a 0.1-0.5 mm screen. In an embodiment, about 90% of the ground vegetable material can fit through a sieve with a 0.1-0.5 mm screen. In an embodiment, all of the ground vegetable material can fit through a sieve with a 0.1-0.5 mm screen. In another embodiment, about 70% or more, of the ground vegetable material can fit through a sieve with a 0.1-0.5 mm screen. In an embodiment, the reduced plant material (e.g., fractionated plant material) can be mixed with liquid at about 20 to about 50 wt-% or about 25 to about 45 wt-% dry reduced plant material (e.g., fractionated plant material). Optimum solids concentration depends on the starch content of the grain or other feedstock and ethanol tolerance of the yeast. The optimum starch content of the mash generally is the amount necessary to produce ethanol to the tolerance of the yeast. With most commercial distillery yeast maximum ethanol concentration is no more than 15%. Therefore if a beginning mash starch concentration exceeds about 30%, the amount over 30% would not be converted to ethanol by the yeast. On a mole weight basis, one mole of glucose or 180 grams converts to two moles of ethanol with a total gram molecular weight of 92 grams (46 grams per mole ethanol) 92/180=0.511. On a weight basis 51% of glucose converts to ethanol at theoretical yield)

The vegetable material can also be fractionated into one or more components. Any starch containing component can be employed in the process. For example, a vegetable material such as a cereal grain or corn can be fractionated into components such as fiber (e.g., corn fiber), germ (e.g., corn germ), and a mixture of starch and protein (e.g., a mixture of corn starch and corn protein). One or a mixture of these components can be fermented in a process according to the present invention. Fractionation of corn or another plant material can be accomplished by any of a variety of methods or apparatus. For example, a system manufactured by Satake can be used to fractionate plant material such as corn.

In one embodiment, the germ and fiber components of the vegetable material can be fractionated and separated from the remaining portion of the vegetable material. In another embodiment, the remaining portion of the vegetable material (e.g., corn endosperm) can be further milled and reduced in particle size and then combined with the larger pieces of the fractioned germ and fiber components for fermenting.

In one embodiment, the vegetable material can be milled to access value added products (such as neutraceuticals, leutein, carotenoids, xanthrophils, pectin, cellulose, lignin, mannose, xylose, arabinose, galactose, galacturonic acid, GABA, corn oil, albumins, globulins, prolamins, gluetelins, zein and the like).

Fractionation can be accomplished by any of a variety of methods and apparatus, such as those disclosed in U.S. Patent Application Publication No. 2004/0043117, the disclosure of which is incorporated herein by reference. Suitable methods and apparatus for fractionation include a sieve, sieving, and elutriation. Suitable apparatus include a frictional mill such as a rice or grain polishing mill (e.g., those manufactured by Satake, Kett, or Rapsco).

The prepared plant material (e.g., fractionated plant material) can be referred to as being or including “raw starch”. The starting plant material is generally processed to produce a mash that has starch in a form more accessible and thus more easily converted than the starting plant material. By “starch” herein is meant any material comprised of the complex polysaccharide carbohydrates of plants, comprised of amylose and amylopectin with the formula (C₆H₁₀O₅)ₓ, wherein X can be any number. In particular, “starch” refers to any plant-based material including, but not limited to, grains, grasses, tubers and roots and more specifically wheat, barley, corn, rye, rice, sorghum, brans, cassava, millet, potato, sweet potato, and tapioca.

The present invention provides processes for converting starch (usually from processed plant material as outlined herein) to sugars that can be fermented by a microorganism such as yeast. This conversion can be carried out by saccharifying the reduced plant material with any of a variety of known saccharifying enzyme compositions.

By “hydrolysis”, “saccharification” or “saccharifying” herein is meant the process of converting starch to smaller polysaccharides and eventually to monosaccharides, such as glucose, with enzymes, e.g., glucoamylase and amylase. Conventional saccharification uses liquefaction of gelatinized starch to create soluble dextrinized substrate which glucoamylase enzyme hydrolyzes to glucose. In the present method, saccharification refers to converting raw starch to glucose with enzymes, e.g., glucoamylase and amylase. According to the present method, the raw starch is not generally subjected to conventional liquefaction and gelatinization to create a conventional dextrinized substrate, although as outlined herein, the enzyme preparations of the invention also find use in convention processes

In one embodiment, saccharification is conducted at a pH of about 6.0 or less, for example, about 3.5 to about 5.0, for example, about 3.5 to about 4.0, and preferably about 3.5.

In the present method, the raw starch is not subjected to conventional liquefaction and gelatinization to create a conventional dextrinized substrate, i.e., “without cooking.”

By “without cooking” herein is meant a process for converting starch to ethanol without significant heat treatment for gelatinization and dextrinization of starch using alpha-amylase. Generally, for the process of the present invention, “without cooking” refers to maintaining a temperature below starch gelatinization temperatures, so that saccharification occurs directly from the raw native insoluble starch to soluble glucose while bypassing conventional starch gelatinization conditions. Starch gelatinization temperatures are typically in a range of 57° C. to 93° C. depending on the starch source and polymer type. In the method of the present invention, dextrinization of starch using conventional liquefaction techniques is not necessary for efficient fermentation of the carbohydrate in the grain.

Saccharifying can be conducted without cooking. For example, saccharifying can be conducted by mixing source of saccharifying enzyme composition (e.g., the enzyme preparation provided herein), yeast, and fermentation ingredients with ground grain (e.g. the “mash”) and process waters without cooking.

In one embodiment, saccharifying can include mixing the processed plant material with a liquid, which can form a slurry or suspension and adding the enzyme preparations of the present invention to the liquid. Alternatively the addition of the enzyme preparation can precede or occur simultaneously with mixing, in any order.

In one embodiment, the reduced plant material (e.g., fractionated plant material) can be mixed with liquid. In one embodiment, the solids concentration is such that the mash starch concentration is 25 to 30% (w/w) to produce a fermented beer with 10 to 15% (w/w) ethanol. As used herein, wt-% of reduced plant material in a liquid refers to the percentage of dry substance reduced plant material or dry solids. In one embodiment, the method of the present invention can convert raw or native starch (e.g., in dry reduced plant material) to ethanol at a faster rate at higher dry solids levels compared to conventional saccharification with cooking. Although not limiting to the present invention, it is believed that the present method can be practiced at higher dry solids levels because, unlike the conventional process, it does not include gelatinization, which increases viscosity.

Suitable liquids include water and a mixture of water and process waters, such as stillage (backset), scrubber water, evaporator condensate or distillate, side stripper water from distillation, or other ethanol plant process waters, as well as nutrient solutions disclosed herein. In one embodiment, the liquid includes water. In another embodiment, the liquid includes water in a mixture with about 1 to about 70 vol-% stillage, about 15 to about 60 vol-% stillage, about 30 to about 50 vol-% stillage, or about 40 vol-% stillage. In addition, solutions suitable as nutrient sources outlined above can be added here as well.

In the conventional process employing gelatinization and liquefaction, stillage provides nutrients for efficient yeast fermentation, especially free amino nitrogen (FAN) required by yeast. The present invention can provide effective fermentation with reduced levels of stillage and even without added stillage. In an embodiment, the present method employs a preparation of plant material (e.g., fractionated plant material) that supplies sufficient quantity and quality of nitrogen for efficient fermentation under high gravity conditions (e.g., in the presence of high levels of reduced plant material). Thus, in an embodiment, no or only low levels of stillage can suffice. Generally, stillage recycle for FAN is necessary in wet mill corn processes where the ethanol feedstock is a highly purified starch with little or no associated protein. When using whole grain in dry mill processes FAN is much less of an issue as the grain protein content is sufficient to support yeast growth and metabolism. However stillage recycle to reduce waste water treatment volume can be important in dry mill ethanol plant designs. The ATSH fermentation process provided herein can take place with or without stillage recycle in dry mill whole grain processes.

However, the present method provides the flexibility to employ high levels of stillage if desired. The present method does not employ conventional liquefaction. Conventional liquefaction increases viscosity of the fermentation mixture and the resulting stillage. The present method produces lower viscosity stillage. Therefore, in an embodiment, increased levels of stillage can be employed in the present method without detrimental increases in viscosity of the fermentation mixture or resulting stillage.

Further, although not limiting to the present invention, it is believed that conventional saccharification and fermentation processes require added FAN due to undesirable “Maillard Reactions” which occur during high temperature gelatinization and liquefaction. The Maillard Reactions consume FAN during cooking. As a result, the conventional process requires adding stillage (or another source of FAN) to increase levels of FAN in fermentation. It is believed that the present process avoids temperature induced Maillard Reactions and provides increased levels of FAN in the reduced plant material, which are effectively utilized by the yeast in fermentation.

Saccharification can employ any of a variety of known enzyme sources (e.g., a microorganism) or compositions to produce fermentable sugars from the reduced plant material (e.g., fractionated plant material). In an embodiment, the saccharifying enzyme composition includes an amylase, such as an alpha amylase (e.g., an acid fungal amylase) or a glucoamylase, and preferably, the enzyme preparation provided herein.

In an embodiment, saccharification is conducted at a pH of about 6.0 or less, pH of about 3.0 to about 6.0, about 3.5 to about 6.0, about 4.0 to about 5.0, about 4.0 to about 4.5, about 4.5 to about 5.0, or about 4.5 to about 4.8. In an embodiment, saccharification is conducted at a pH of about 4.1 to about 4.6 or about 4.9 to about 5.3. Preferred pH is 3.5 to 4 for simultaneous hydrolysis and fermentation at ambient temperature. The initial pH of the saccharification mixture can be adjusted by addition of, for example, ammonia, sulfuric acid, phosphoric acid, process waters (e.g., stillage (backset), evaporator condensate (distillate), side stripper bottoms, and the like), and the like.

The enzyme preparation provided in the present invention can be used as a replacement for expensive purified commercially available in conventional multiple-step process such that described in U.S. Patent Publication Nos: 20040234649, 20050233030, 20050239181, 20070036882, and 2007003267, herein all incorporated by reference. These publications relate to a process for converting raw starch to at least 15% ethanol with cooking. They disclose the methods of grinding the grain to a fine composition to allow a higher slurry concentration, and cooling the fermentation mash to allow the beer to reach at least 15% ethanol. There are few types of yeast that tolerate over 15% of ethanol at 30° C. The reduction in fermentation temperature increases the yeast tolerance to ethanol, allowing the beer concentration to reach 15%. Most yeasts are more tolerant of ethanol concentrations at lower temperatures, but the rate of conversion of sugar to ethanol decreases. Most yeast has an optimum performance around 30° C. Therefore, lower operating temperature may slow the fermentation process down In addition, cooling the fermentation mash below 30° C. requires more energy and more equipment. However, in some embodiments, the present invention can be carried out at lower temperature to produce higher percentage of alcohol.

The sugar from starch hydrolysis is subjected to fermentation to produce ethanol. After the saccharification step is completed, the fermentable sugars are added to yeast where fermentation begins. Thus, the steps of starch hydrolysis and fermentation can be carried out separately. Alternatively, the steps of starch hydrolysis and fermentation can be carried out simultaneously. It is also possible to have overlapping steps of hydrolysis and fermentation. For example, the fermentation step can be initiated after the hydrolysis step starts, but before the hydrolysis is completed. This simultaneous saccharification and fermentation allows for higher concentrations of starch to be fermented.

### IV. Integrated Process

Because the grain mash is not cooked at a high temperature as in conventional ethanol processes, it is possible to use water containing ethanol to form the grain mash. If water with ethanol were used to form the grain mash and then cooked in the conventional mash cooking process, the ethanol would vaporize and be lost.

In some embodiments, the fermentation beer from the cellulose ethanol production is integrated with the ATSH process, which is described above.

Briefly, in this process, the cellulose fermentation beer is mixed with grain to form a mash and enzyme and yeast are added in a simultaneous hydrolysis and fermentation of the starch. The process takes place entirely in a single vessel at fermentation temperature of 25° C. to 40° C. and reaches a final ethanol concentration of up to 15% w/w. Alternatively, the cellulose ethanol beer could be mixed with starch and no cook starch enzymes prior to the addition of yeast. One or more vessels can be used. The hydrolysis step can precede the fermentation step. The whole process takes place at temperatures below the boiling point of ethanol (78.4° C.).

At the completion of fermentation the dilute ethanol “beer” from the cellulose hemicellulose hydrolysis fermentation is used as the process water to make a grain mash. The dilute beer may be transferred to separate fermentation vessels or ground grain may be added directly to the dilute ethanol beer in the cellulose fermentation tank. Grain mash will typically contain up to 40% w/w solids with the solids content determined by the starch content necessary to achieve 10% to 12% w/w ethanol from the starch with near theoretical conversion of starch to ethanol

The mash from the above step can be treated with any enzymatic “no cook” or reduced temperature starch hydrolysis process to convert the starch to fermentable sugar (glucose or possibly soluble short chain glucose polymers that can be fermented to ethanol by some microbes). The starch hydrolysis must be carried out at temperatures below the boiling point of ethanol or under conditions in which the ethanol is not vaporized and lost from the process.

Generally, the mash pH is adjusted from 3.5 to 4.5 and temperature from 25° C. to 40° C. then no cook “ATSH” enzyme preparation and yeast are added to initiate a simultaneous starch hydrolysis and fermentation. The composition of ATSH enzyme and method of preparation is described in more detail above. The hydrolysis fermentation typically requires 36 to 72 hours and reaches a final ethanol concentration of 10 to 12% w/w. This high concentration beer containing ethanol from both the lignocellulosic and starch feedstocks is then distilled using standard distillation equipment and processes.

### V. System for Producing Ethanol

In one aspect the invention relates to a system that produces ethanol.

In general, the systems comprise bioreactors to carry out the integrated ethanol production system described herein.

In an embodiment, the invention relates to a system that produces ethanol. A diagram of the system in shown in FIG. 5. The present system can include a saccharification unit 1, a fermentation unit 2, a distillation unit 3 and a dryer unit 4.

The saccharification unit 1 can be any of a variety of apparatus suitable for containing or conducting saccharification. The saccharification unit 1 can be, for example, a vessel in which cellulosic material can be converted to a sugar which can be fermented by a microorganism such as yeast. The saccharification unit 1 can be configured to maintain a saccharification mixture under conditions suitable for saccharification. The saccharification unit 1 can be configured to provide for the conversion of cellulosic material with the addition of enzymes. In one embodiment, the saccharification unit 1 is configured for mixing cellulosic material with a liquid and adding a saccharifying enzyme composition to the liquid. In another embodiment, the saccharification unit 1 is configured for saccharification at a variety of pHs and temperatures, but preferably at a pH of 4.5 to 5.0 and at a temperature of about 30 to about 50° C., preferably about 45° C.

The fermentation unit 2 can be any of a variety of apparatus suitable for containing or conducting fermentation. The fermentation unit 1 can be, for example, a vessel in which sugar from cellulosic material can be fermented to ethanol. The fermentation unit 2 can be configured to maintain a fermentation mixture under conditions suitable for fermentation. In one embodiment, the fermentation unit 2 can be configured for fermenting through use of a microorganism, such as yeast. In another embodiment, the fermentation unit 2 can be configured to ferment a saccharification mixture. In yet another embodiment, the apparatus can employ any variety of yeasts that yields a commercially significant quantity of ethanol in a suitable time. Yeast can be added to the apparatus by any of a variety of methods known for adding yeast to a system that conducts fermentation. The fermentation unit 2 can be configured for fermentation for about 24 to 96 hours at a temperature of about 20 to about 40° C.

The saccharification unit 1 and the fermentation unit 2 can be a single, integrated apparatus. In one embodiment, this apparatus is configured to provide higher temperatures early on during simultaneous conversion of cellulosic material to sugars and fermentation of those sugars. In an embodiment, this apparatus is configured to provide lower temperatures later during the simultaneous saccharification and fermentation. The apparatus also may utilize the reagents and conditions described above for saccharification and fermentation, including enzymes and yeast.

The distillation unit 3 can be any of a variety of apparatus suitable for distilling products of fermentation. The distillation unit 3 can be, for example, configured to recover ethanol from the fermentation mixture (“beer”). In one embodiment, the fermentation mixture is treated with heat prior to entering the distillation unit 3. In another embodiment, fractions of large pieces of germ and fiber are removed with a surface skimmer or screen prior to or after entering the distillation unit 3.

The dryer unit 4 can be any of a variety of apparatus suitable for drying solids remaining after distillation (and optional centrifugation, for example, in a centrifuge system). In an embodiment, the dryer unit 4 is configured to dry recovered solids, which can result in production of distiller's dried grain. After the distillation system separates the ethanol from the beer, recovered solids remain. These recovered solids can then be dried in the dryer unit 4. This produces distiller's dried grain and/or distiller's dried grain plus solubles. In one embodiment, the dryer unit 4 can be or include a ring dryer. In another embodiment, the dryer unit 4 can be or include a flash dryer. In yet another embodiment, the dryer unit 4 can be or include a fluid bed dryer.

The distillation step is identical to conventional ethanol process with one exception. The beer from fermentation process is usually lower in ethanol than from a starch process. The lower concentration is a result of the lower solids concentration in the original mash. The concentration of straw mash typically is 10% w/w; it is generally difficult to keep straw suspended in a slurry at concentrations above 10%. Because of the more dilute beer, distillation costs are higher. However the higher distillation costs are offset by lower feedstock costs.

The process of cellulose hydrolysis/fermentation and starch hydrolysis/fermentation can be carried out in separate biochambers known in the art.

In some embodiments, the beer generated from cellulose hydrolysis is introduced into a bioreactor that is used for starch hydrolysis/fermentation. Preferably, the facilities for cellulose and starch hydrolysis/fermentation are located closely to reduce transportation cost.

Corn stover, wheat and switchgrass contain 37-40% cellulose, 26-30% hemicellulose and 15-20% lignin, Sun Grant Initiative, North Central Center, South Dakota State University. 95% of the cellulose and approximately 65-85% of the hemicellulose can be converted to ethanol, depending on the feedstock, delignification process, enzymes and yeast used. The lignin or residual biomass can provide electricity and process heat to both reduce costs and improve the net energy value of ethanol production. Dry lignin has a High Heating Value (HHV) of 10,000 to 11,000 BTU/lb while hemicellulose has a HHV of 8000 BTU/lb. White, R. H. Wood and Fiber Science, 19(4), 1987, pp. 446-452.

In some embodiments, the total volume of water heated and distilled in an integrated plant will be similar to the volume of water in a conventional starch process if the cellulose beer is used as the starch fermentation water and no additional cooking is necessary. For example, an integrated cellulose starch ethanol plant producing 110 M gallons of ethanol/year could produce 82.5 M gallons of ethanol from starch and 27.5 M gallons of cellulose based ethanol (75% of the ethanol from starch in a 12% ethanol beer and 25% from cellulose/hemicellulose in a 3% ethanol beer) would use 325,000 tons of corn stover, generating 61,750 tons of lignin with a heating value of 8981 BTU/gal ethanol produced. The thermal value of the lignin is equivalent to a 29% reduction from the 31,000 BTU's/gal energy requirement estimated for a conventional plant. Increasing the percentage of lignocellulose feedstock would further reduce overall energy consumption and reduce feedstock costs.

In some embodiments, synthesis gas, a mixture of CO and H₂, is produced by biomass gasification. Gasification is more flexible, efficient and environmentally compatible than direct combustion. Synthesis gas used for electricity generation with the waste heat used to meet process energy requirements could potentially reduce annual energy costs and provide the integrated plants electrical needs. Surplus electricity, depending on the amount of biomass available for gasification, could be sold.

Corn stover, cereal straws, and strawlike biomass fast growing plants contain more ash, potassium and chloride than wood. Wood grows slower and usually contains less than 1% ash (excluding the bark). Gasification technologies for wood are fairly well developed. There are a number of new gasification technologies for agricultural residues that are developed and can be used as a process component in an integrated cellulose starch fermentation facility.

Anaerobic digesters are frequently being used when starch ethanol plants are built close to feedlots or dairies. Wet distillers grains are feed to the cattle and the manure from the cattle is used in an anaerobic digester to generate power for the ethanol plant. Anaerobic digesters can also be used to convert residual biomass (lignin, distillers grains, unconverted hemicellulose) to methane gas. The methane gas is used in a gas fired turbine to generate electrical power. The waste heat is used in the ethanol production process.

The examples provided herein are for illustration purposes only and are in no means to limit the scope of the present invention. Further, all references cited herein are incorporated by reference for all the relevant contents therein.

### EXAMPLES

### Example 1

### Selection of Trichoderma Strain

A number of strains have been tested, including: T. reesei ATCC 56765 (RUT C30), 13631, 24449, 26920, 26921, NRRL 11236, 11480, and 11485; Aspergillus niger ATCC 52172; A. versicolor ATCC 52173 and A. terrus ATCC 52430. All strains tested produced some level of measurable cellulase activity. Of them, ATCC 56765 was selected as the most consistent and highest concentration producer of multiple cellulase and hemicellulase activities and the strain which consistently gave the highest yield in standardized hydrolysis and hydrolysis fermentation of alkali pretreated barley straw.

Selected strains of T reesei were grown in solid substrate culture for further comparison of cellulase production. T. reesei strains were obtained from the ATCC and cultured on PDA agar. Strains were grown in solid substrate culture according to the methods described in example 2. Overall cellulase activity was assayed according to the standardized straw hydrolysis assay also described in Example 2. The results are shown below.

| Strain | Glucose concentration mg/ml at 24 hours |
|---|---|
| ATCC 58351 | 9.0 |
| ATCC 58352 | 8.9 |
| ATCC 58353 | 7.1 |
| ATCC 60787 | 14.3 |
| ATCC 56765 | 22.3 |

ATCC 56765 is the strain selected for optimal SSC cellulase production. Other strains do produce cellulase when grown in the SSC process as described. This experiment tested strains grown in the same experiment under equivalent conditions. Seven other strains of T. reesei as well as strains of Aspergillus niger, A. versicolor, A. phoenicis and A. terreus have been tested in separate experiments grown on substrates with varying ratios of components. Although not directly comparable, results of standardized straw assay and selective substrate enzyme assays do demonstrate production of cellulase by multiple strains when grown in the SSC process. In the standardized straw hydrolysis assay 24 hour glucose concentration ranged from 4 to 18 mg/ml. with filter paper assay of 20 to 100 units per gram.

### Example 2

### Production of Enzyme Composition Using Solid State Culture

Trichoderma reesei, ATCC 56765 was maintained on PDA agar slants at 4° C. Inoculum cultures were prepared by transferring cells and spores from PDA agar slant to broth containing in grams per liter: NH₄SO₄ 2.8; KH₂PO₄ 4.0; MgSO₄ 0.6; Urea 0.6; CaCl 0.3; Yeast extract 1.0; soy peptone 1.0; Glucose 10.0; trace elements MnSO₄, FeSO₄, CUSO₄ all less than 0.001. Inoculum culture was grown at 30° C. for 48 hours and used to inoculate solid culture media. Solid culture media contained in grams per kg dry weight: Corn cob beeswing pith and chaff (BPC) 450, barley flour 350, straw (2 mm) 250, wheat germ 250. Dry ingredients were blended and wetted with two liters of salts solution above less glucose per kg of dry substrate, mixed and autoclave at 120° C. 15 psi for 30 minutes then cooled. Substrate was inoculated at 100 ml inoculum culture per kg dry weight substrate equivalent. Inoculated substrate was incubated in a column reactor, 6 inches in diameter by 24 inches long with caps at both ends equipped for inlet and outlet air flow. Columns were aerated at 50 cc/minute air at 90% RH and held at a constant temperature of 30° C. Culture was incubated for 10 days then removed from the column, dried under a flow of dry air at about 25° C., ground in a mill and assayed for enzyme activity by the following methods.

Filter paper assay: 0.1 gram enzyme was added per one filter paper disc. The assay was carried out for two hour at 50° C. The assay was stopped with the addition of DNS reagent, and boiled for 10 min. The results were read at OD 550 nm. One unit equals 1 mg as total reducing sugar.

CMC (carboxymethyl cellulose) assay: 1.0 ml 1% CMC was added to 1.0 ml enzyme water extract (1:1000 dilution) and incubated for 30 minute at 50° C. The reaction was stopped with DNS reagent. One unit equals 1 mg as total reducing sugar.

Cellulose Azure assay: 2 ml of 1% cellulose azure (Calbiochem) was added to 1.0 ml enzyme water extract (1:1000 dilution) and incubated for one hour at 50° C. The reaction was stopped with 1.0 ml 40% acetic acid, and centrifuged. The supernatant is used for OD reading at 595 nm. The units is expressed as the change of OD595 in one hour.

Xylanase assay: 1.0 ml of 0.5% Larchwood xylan was added to 0.5 ml of enzyme water extract (1:1000 dilution) and 0.5 ml buffer, and incubated for 10 minutes at 50° C. The reaction was stopped with addition of DNS reagent. One unit equals 1.0 mg as total reducing sugar, xylose standard.

Standardized straw assay; 1.0 grams alkali pretreated and washed grain straw in 20 ml buffer was autoclaved, and cooled. 0.1 gram enzyme was added and was incubated at 50° C. for 24 hours, and was assayed as glucose or as total reducing sugar. 25 mg/ml glucose represents 50% hydrolysis of pretreated straw.

Dilute alkali pretreatment: 10% straw in 1% NaOH was autoclaved for 15 minutes, and centrifuged. The solids was washed once in 10 volumes of water. The straw solids was recovered and air dried.

All assays was conducted in 0.2M Na Acetate buffer, pH 4.8. DNS assay was conducted according to the method of Miller G L. Anal. Chem. 31:426-429 (1959). As modified, 0.5 ml of sugar solution or hydrolysate diluted as appropriate is incubated with 1.5 ml dinitro salacyclic acid reagent in a boiling water bath for 10 minutes, cooled and read on a spectrophotometer at 550 nm. Total reducing sugar is determined by comparison to a glucose standard.

Results are shown in Table 1. The data compares solid culture enzyme preparation labeled SSC with a commercial cellulase Novozymes Celluclast. 1.5 L was assayed on an equal weight basis by the same methods.

TABLE 1
Selective Substrate Assays of SSC Enzyme Preparations
Assay Substrate

| | Filter | | Cellulose | | Straw |
|---|---|---|---|---|---|
| | Paper | CMC | Azure | Xylan | Gulucose |
| | units/gr | units/gr | ΔOD₅₉₅ | units/gr | mg/ml |
| SSC | 152 | 118 | 0.263 | 102 | 25 |
| Novo | 144 | 109 | 0.223 | — | 22.4 |

MMP enzyme bench scale production test 500 gram SSC Novo Celluclast 1.5 L cellulase from Trichoderma reesei

### Example 3

### Cellobiase Activity in Solid Culture Enzyme Preparations

One advantage of the composition of the enzyme produced using solid culture technology of the present invention is production of cellobiase activity in the same culture as the endoglucanase and cellobiohydrolase activities. The cellobiase converts cellobiose to glucose which can be fermented by common yeast strains. Enzyme preparations produced according to the method described in example 2 above and assayed for cellobiase activity.

Assay procedure: A solution of cellobiose 2 mg/ml was treated with 10% w/w solid culture derived enzyme and incubated at 30° C. for 30 minutes. The enzyme treatment was compared to the same solution of cellobiose incubated without addition of enzyme. After incubation tubes were assayed for total reducing sugar by the DNS method. This method assays only the reducing end of the molecule measures the two glucose residue cellobiose molecule as a single reducing sugar. If cellobiose is hydrolyzed to monomeric glucose molecules the reducing sugar will increase as both glucoses now react with the DNS reagent. Incubation of the cellobiose solution with solid culture enzyme increased total reducing sugar concentration from 2 mg/ml to 3.5 mg/ml indicating nearly complete conversion of cellobiose to glucose.

### Example 4

### Pilot Scale Solid Substrate Culture for Cellulase/Hemicellulase Enzyme Preparation

The four component substrate described in example 1 was prepared in lots of approximately 20 kg, wetted with the nutrient solution described in example 2. The moist substrate was placed in autoclave bags and autoclaved for approximately one hour at 125° C., cooled and inoculated at 10% v/w of a 48 hour culture of T. reesei ATCC 56765 grown in the broth medium described in example 1. The inoculated substrate was placed on screen trays in a commercial solid culture incubation chamber and incubated for 10 days at about 30° C. under a constant flow of air at 90% RH. After 10 days the cultures were dried and ground and used as the cellulase enzyme preparation as describe in example 5 below.

### Example 5

### Summary Process Description

Pulverized straw was treated with 1% NaOH at 80° C. and 9 psi. The wet straw was passed through a screw press to remove the “black liquor” (black liquor is a caustic solution containing solubilized lignin and other materials). The wet pulp was washed and passed through a second and then third screw press to remove additional solubles and to help adjust pH. The squeezed pulp was fed to a 200 gallon stirred fermentation tank. Water, enzyme, yeast and acid were added to the fermentation tank. The fermentation tank was maintained at approximately 20° C., or room temperature. Samples were collected from the fermentation tank at 66 hours, 72 hours, and 86 hours. The fermentation liquid was pumped through a screw press to separate residual solids from the liquid. The separated liquid was used as the water for a starch fermentation tank. The solids, which correspond to distillers grains from a corn fermentation process, were disposed off. The solids contain fiber and also some protein as a result of the yeast cells. They have potential application as a livestock feed.

The pulverized grass straw was used as a stand-alone feedstock in a simultaneous hydrolysis and fermentation process to produce a beer containing 3.46% ethanol or 83 gallons ethanol per dry weight ton of delignified grass straw. The milled barley straw was used as a stand-alone feedstock to produce a beer containing 5% ethanol or 100 gallons ethanol per dry weight ton of delignified straw. The pulverized grass straw was used in a 10% w/w fermentation slurry while the milled barley straw was used in a 12% w/w fermentation slurry.

The solids were separated from the liquid using a screw press. The separated water, containing 3.46% w/w ethanol from the grass straw, was used as fermentation water to make a 30% slurry with ground barley. Acid and yeast were added to the fermentation liquid. Temperature was maintained at approximately 24° C. After 72 hours, the fermentation broth (beer) contained 13.74% ethanol. A typical ATSH fermentation of a 30% barley slurry results in 12.375% ethanol.

Each step in the process is discussed below. Grass seed straw and barley straw were tested in separate experiments using same basic process.

### Example 6

### Straw Preparation

Straw bales composed of annual rye, perennial rye, and tall fescue straw were obtained from growers in the Willamette Valley of Oregon. Bales of barley straw were obtained from a rancher in Beaverhead County. The pilot facility was equipped with straw chopper that was initially used to cut the straw into pieces approximately one inch long. The large particle size required more severe pretreatment, i.e. higher temperature, more residence time and higher concentrations of NaOH. In addition to being more expensive, it increased the cost of the capital equipment because of the higher caustic concentrations and higher operating pressure used to achieve the higher temperature.

It was determined that it was more economical to mill the particles to a finer size and reduce energy and chemical costs. An air swept pulverizer was used to mill the grass straw for the fermentation demonstration. 99% of the milled straw would pass US Standard 40 mesh (425 micron) screen and 80% passes 60 mesh (250 micron). The various types of grass straw were blended together in equal weight fractions.

A commercial hay processor consisting primarily of a hammer mill, conveyor, and hopper was used to grind the barley straw. 85% of the straw would pass US Standard 20 mesh screen. Less than 5% would pass a 40 mesh screen. While the hammer mill resulted in larger particles than the air swept pulverizer, the particles were still much smaller than those from the straw chopper that was part of the delignification pilot plant.

### Example 7

### Delignification

Pulverized straw (175 lbs/batch) was mixed with water to form a 10% w/w slurry. NaOH was added to the water/straw mixture to 1% w/v final concentration.

The straw slurry was pumped into a stainless steel pressure tank where it was heated with direct steam injection. It took approximately 45 minutes to heat the tank to 80° C. The tank pressure was approximately 8 psi. For the grass straw, the tank was held at such temperature and pressure for two hours. The barley straw was held at 100° C. and 12 psi pressure for eight hours. At that time, the steam pressure in the tank was used to convey the straw slurry at 3.5 gpm to the first screw press. It took approximately one hour to empty the tank. The grass straw residence time in the tank varied from 2 hrs 45 minutes to three hours 45 minutes. The barley straw residence time varied from 8 hours 15 minutes to 9 hours 15 minutes.

The screw press separated the soluble lignin from the residual cellulose and hemicellulose. The resulting black liquor was neutralized with sulfuric acid and disposed of. A commercial plant could reuse a portion of the black liquor, precipitate out the lignin, and use the lignin as a fuel source.

Steam explosion, acid hydrolysis, solvent extraction, ammonia explosion, and high temperature with oxygen injection are other techniques that can be used to remove lignin from straw.

### Example 8

### Pulp Preparation

The pilot tests used screw presses to separate black liquor and wash the delignified straw pulp. The pulp from the first screw press was washed with fresh water and then passed through two additional screw presses to remove any additional solubles. The pH of the wash water removed from the additional screw presses varied from 7.5 to 7.8, indicating most of the caustic was removed with the black liquor (pH 10). One advantage of screw presses over screens is the better water removal. A commercial facility would only require two screw presses rather than the three that were used in the pilot facility. Centrifuges or other de-watering devices could also be used instead of screw presses. The delignified, washed straw pulp was fed to the fermentor.

### Example 9

### Pilot Ethanol Production

Pilot tests of ethanol production used 200 gallon plastic fermentation tanks equipped with agitators. The tanks were used for simultaneous hydrolysis and fermentation. The washed (delignified) straw pulp was suspended at 10% w/w or 12% w/w in water in the tank. The wet barley straw pulp and wet grass straw pulp were added at the same weight, but subsequent analysis showed the barley straw pulp was at a lower moisture content, resulting in a higher slurry concentration. Acid was added to adjust the pH to 4.8. Solid substrate culture cellulase enzyme preparation was added at 5% w/w solids. Conventional alcohol distillery yeast was added at the rate of one pound per 1000 gallons per manufacturer's instructions. Hydrolysis and fermentation occurred at room temperature, approximately 20° C. The fermentation broth temperature increased to 24° C. approximately 16 hours into the grass straw fermentation. The temperature was less than optimum for the yeast (30-35° C.) which may have slowed the fermentation process. Samples were taken at 66, 72, and 86 hours. Samples were refrigerated and then shipped to Energy Laboratories for ethanol analysis by gas chromatography with results expressed as % ethanol volume/volume. Sample results showed an overall yield of 83 gallons ethanol per dry ton of delignified straw. Alcohol concentration peaked around 72 hours.

The beer from the grass straw fermentation was used as fermentation water for a barley grain fermentation using the ATSH process. The solids were separated from the beer using a screw press. The separated water, containing 3.46% v/v ethanol from the grass straw, was used as fermentation water to make a 30% w/v slurry with ground barley. ATSH enzyme at a loading of 2.5% of barley weight, acid and yeast were added to the fermentation liquid. Temperature was maintained at approximately 24° C. After 72 hours, the fermentation broth (beer) contained 13.74% v/v ethanol. A typical ATSH fermentation of a 30% w/v barley slurry with barley at 50% starch results in a theoretical ethanol yield of 10.6% v/v ethanol. A mass balance around the theoretical starch ethanol yield plus the measured ethanol from the cellulose fermentation would predict a final ethanol concentration of 14.06% v/v (3.46+10.6=14.06) The final measured ethanol concentration of 13.74% v/v represents about 97% of the theoretical yield with 24.6% of the ethanol derived from the cellulose.

### Example 10

### General Example

Step 1. A lignocellulosic material such as straw, corn stover, switch grass, sugar cane stalks, oat hulls, wheat hulls, or wood is pretreated to disrupt or disassociate the hemicellulose. Pretreatment may be any chemical or mechanical means or combination of chemical and mechanical pretreatment including acid treatment, alkaline treatment, steam explosion, solvent extraction, oxygen injection, hot water, grinding, milling etc.

Step 2: The pretreated material is further processed as a liquid or liquid/solid slurry. The pretreated lignocelluloses material may or may not be separated into soluble and insoluble fractions

Step 3. The insoluble fractions or combined fractions, containing cellulose are treated with enzymes to convert the cellulose to sugar (hydrolysis).

Step 4. The sugar is fermented to ethanol using yeast, a combination of yeast, or bacteria. Steps 3 and 4 can also be combined in a single process step for a simultaneous hydrolysis and fermentation step. If the process stream has been separated into soluble and insoluble fractions, the soluble fraction may contain sugars that can be fermented to alcohol without the addition of enzymes.

Step 5: The fermentation liquid (beer) from the lignocellulosic streams processed separately or together will contain 1-8% ethanol. This fermentation beer from the lignocellulose fermentation will be mixed with a ground or milled starch component. Typically the starch will originate with corn, wheat, barley but could be any starch.

Step 6: Starch degrading enzymes will be added to the mixed stream containing 1-8% ethanol and starch. The mixed stream containing starch may be heated prior to or after the addition of the enzymes, but at no time will the temperature of the mixed stream exceed 78° C. or the boiling point of ethanol (78.4° C. at atmospheric pressure). In the preferred process, the mixed stream is never heated above 35° C.

Step 7. Yeast or bacteria are added to the process stream used to ferment the sugar, converting the starch sugars to ethanol.

Steps 6 and 7 may be combined into a single simultaneous hydrolysis and fermentation step or performed sequentially. The resulting fermentation beer will have an ethanol alcohol content that is the sum of the ethanol from the lignocellulose fermentation and the starch fermentation. Typically this will be 12-15% ethanol.

### Example 11

### Example from One Pilot Plant Test

Step 1: Pretreatment: Straw was ground to allow 90% of the straw to pass a 20 mesh screen. The straw was mixed with water to form a 10% weight to weight slurry. 1% sodium hydroxide was added to the slurry and the slurry was heated to 80° C. for three hours. The alkaline pretreatment solubilized a portion of the lignin. The solubilized lignin was separated from the insoluble fraction which was primarily cellulose and hemicellulose but did contain small amounts of other residues from the original straw material, including lignin, fiber, ash, silica, etc.

Step 2: The insoluble fraction was mixed with water to make a 10% w/w solids slurry. The slurry pH was adjusted to 4.8 by adding sulfuric acid.

Steps 3 & 4: The slurry was pumped to a fermentation tank where enzymes with cellulose activity (cellulases) were added and a conventional brewers yeast was added. These were Solid Substrate Culture cellulase enzyme preparation but could have been a mixture of several commercial enzymes with different cellulose and hemicellulose degrading activities. The yeast was a conventional brewers yeast that converted six carbon sugars to ethanol, but did not convert 5 carbon sugars. A different yeast or bacteria could have been used in a separate process step to convert the five carbon sugars to ethanol, or there may be some fermenting organisms capable of converting both the five and six carbon sugars to ethanol. The fermentation process was controlled between 25-35° C. for 72 hours. Ideally, the fermentation temperature would be controlled at the optimum temperature for the yeast in this simultaneous hydrolysis and fermentation step.

Step 5: The beer from the lignocellulose fermentation, containing 2.2% ethanol plus five carbon sugars, and residual biomass was mixed with ground barley to form a 30% w/v slurry

Steps 7&8: The slurry was pumped to a fermentation tank where ambient temperature starch enzyme (ATSH enzyme) and yeast were added. The fermentation process was controlled at 25-35° C. for 72 hour. At the end of 72 hours, the ethanol concentration in the beer was 14.7% v/v. A mass balance around the starch and alcohol in the system would predict alcohol concentrations around 14.6%. The difference between 14.6% and 14.7% may be a result of experimental error or a small difference in starch content of the barley feedstock.

### Example 12

### Using Acid Pretreatment and Genencor Enzymes

Step 1: Straw ground to allow 90% of the straw to pass a 20 mesh screen. The straw would be mixed with water to form a 10% weight to weight slurry. A dilute sulfuric acid would be added to the slurry and the slurry was heated. The concentration of the acid, the temperature and the holding time vary from process to process.

Step 2: The acid pretreatment step can hydrolyze the hemicellulose to create soluble five carbon sugars. The solubilized sugars (hydrostate) may be separated or left with the insoluble cellulose fraction.

Step 3: If the streams are left as combined streams, Genencor's cellulase enzymes are added to the entire process mixture, typically at temperatures less than 45° C. (Genencor and Novozyme are working at increasing temperature tolerance and pH range of their enzymes. Currently enzyme stability decreases rapidly at temperatures above 45° C.) If the hydrostate is separated from the insoluble cellulose, the enzymes are added to the cellulose slurry and not to the hydrostate.

Step 4: The combined stream or individual streams undergo a fermentation process using yeast or bacteria. Typically the fermentation process takes 1-3 days, and may be a separate fermentation for the five and six carbon sugars or a single fermentation depending on the organism used. The beer from the fermentation process will contain 1-8% ethanol.

Step 5: The beer from the lignocellulose fermentation process containing 1-8% beer will be mixed with ground starch, typically corn starch. The slurry will contain 20-35% solids depending on the alcohol concentration in the lignocellulose fermentation beer. The starch solids will be added in a proportion to provide a final beer concentration (starch beer plus lignocellulose beer) of approximately 15%. 15% ethanol is the upper alcohol tolerance of most conventional brewers yeasts.

Step 6: The starch slurry will be heated to 68-70° C. to help solubilize the starch. Genencor has some starch hydrolysis enzymes that work at temperatures approaching the gelatinization temperature of the starch. Starch degrading enzymes may be added before or after the heating step. The resulting “mash” is further cooled and more enzymes added.

Step 7: The mash or liquid slurry from the enzyme hydrolysis step is mixed with yeast in a fermentor. The fermentation process takes 1-3 days. The beer from the fermentation process will contain approximately 15% ethanol.

What is claimed is:

1. A method of producing ethanol, comprising:
(1) providing a fermented beer from a cellulose fermentation;
(2) mixing said fermented beer with a starch source to form a mixed mash;
(3) mixing said mixed mash with a starch enzyme composition and yeast; and
(4) incubating for a period of fermentation time at a first temperature between 20 to 78° C. to produce ethanol.

2. The method according to claim 1, wherein said starch enzyme composition is produced with the Aspergillus phoenicis deposited as NRRL 50090.

3. The method according to claim 1, wherein said starch enzyme composition is produced by growing a fungus on a solid state substrate.

4. The method according to claim 1, wherein said cellulose fermentation comprises:
(a) providing a cellulostic feedstock that is adjusted to pH 4 to 6;
(b) mixing said cellulostic feedstock with a cellulase enzyme composition and yeast; and
(c) incubating for a period of fermentation time under a second temperature between 20 to 40° C. to produce said fermented beer.

5. The method according to claim 4, wherein said cellulase enzyme composition is produced with the Trichoderma reesei strain ATCC 56765.

6. The method according to claim 4, wherein said cellulase enzyme composition is produced by growing a fungus on a solid state substrate.

7. The method according to claim 4, wherein said incubating is conducted at pH 4.5 to 5.0.

8. The method according to claim 1, wherein said fermented beer contains less than 7% (w/w) ethanol.

9. The method according to claim 8, wherein said fermented beer contains about 2.5 to 6% (w/w) ethanol.

10. The method according to claim 1, wherein said first temperature is about 35° C.

11. The method according to claim 1, wherein said method is carried at a temperature lower than the boiling point of ethanol.

12. The method according to claim 1, wherein said ethanol produced is less than 15% (w/w).

13. The method according to claim 1, wherein said method further comprising collecting said ethanol.

14. The method according to claim 13, further comprises distilling said ethanol.

15. The method according to claim 1, wherein said starch source is un-gelatinized.

16. The method according to claim 1, wherein said starch source is gelatinized.
